(19) United States
(12) Patent Application Publication
(10) Pub. No.: US 2008/0193988 A1
(43) Pub. Date: Aug. 14, 2008

(54) Process for the Preparation of Sulfonamide Derivatives

(76) Inventors: Iain Robert Gladwell, Kent (GB); Pieter David De Koning, Kent (GB); Ian Brian Moses, Kent (GB); Alan John Pettman, Kent (GB); Nicholas Murray Thomson, Kent (GB)

Correspondence Address: PFIZER INC., PATENT DEPARTMENT, MS8260-1611, EASTERN POINT ROAD, GROTON, CT, 06340, US

(21) Appl. No.: 11/995,988

(22) PCT Filed: Jul. 10, 2006
(86) PCT No.: PCT/IB2006/001958

§ 371 Date: Jan. 17, 2008

Related U.S. Application Data
(60) Provisional application No. 60/700,098, filed on Jul. 18, 2005.

(51) Int. Cl. C12P 7/62; C07C 319/00; C07D 303/08

(52) U.S. Cl. 435/135; 564/162; 549/551

(57) ABSTRACT

The invention relates to a process for the preparation of compounds of formula (I) wherein Q¹ is a group selected from formulae (II) & (III) and a group *-NR⁶-Q²-A or, if appropriate, their pharmaceutically acceptable salts and/or isomers, tautomers, solvates or isotopic variations thereof, as well as intermediates used in said process.

The invention relates to a process for the preparation of compounds of formula (I)

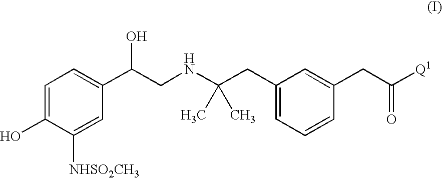

wherein Q¹ is as defined hereafter; or, if appropriate, their pharmaceutically acceptable salts and/or isomers, tautomers, solvates or isotopic variations thereof, as well the intermediates used in said process, or, if appropriate, their salts and/or isomers, tautomers, solvates or isotopic variations thereof.

The compounds of formula (I) are agonists of the β2 receptors, which are particularly useful for the treatment of β2-mediated diseases and/or conditions, by showing excellent potency, in particular when administered via the inhalation route.

The invention relates to a process for the preparation of compounds of formula (I),

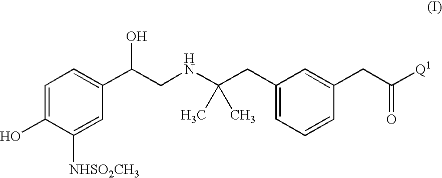

wherein Q¹ is a group selected from:

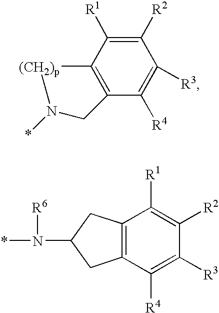

and a group *-NR⁶-Q²-A, wherein the symbol * represent the attachment point to the carbonyl group, p is 1 or 2, Q² is a C₁-C₄ alkylene optionally substituted with one hydroxy group, R⁶ is H or C₁-C₄ alkyl and A is pyridyl optionally substituted with OH, C₃-C₇ cycloalkyl optionally substituted with OH, or a group

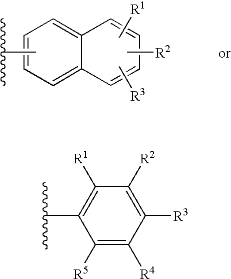

wherein R¹, R², R³, R⁴ and R⁵ are the same or different and are selected from H, C₁-C₄ alkyl OR⁷, SR⁷, halo, CN, CF₃, OCF₃, COOR⁷, SO₂NR⁷R⁸, CONR⁷R⁸, NR⁷R⁸, NHCOR⁷ and phenyl optionally substituted with 1 to 3 groups selected from OR⁷, halo and C₁-C₄ alkyl, wherein R⁷ and R⁸ are the same or different and are selected from H or C₁-C₄ alkyl;
or, if appropriate, their pharmaceutically acceptable salts and/or isomers, tautomers, solvates or isotopic variations thereof.

The invention relates to a process for the preparation of compounds of formula (I)

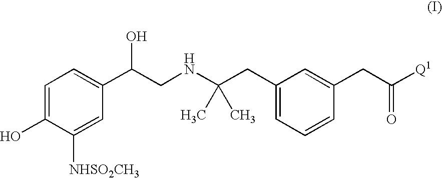

wherein Q¹ is as defined above, comprising the use of a compound of formula

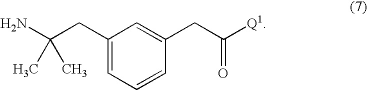

Preferably, the above process comprises the step of reacting said compound of formula (7) with a compound of formula (5),

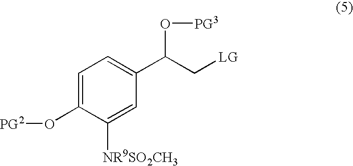

or a compound of formula (6)

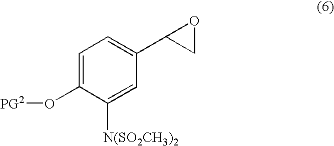

wherein PG² is a suitable phenol protecting group, PG³ is a suitable hydroxyl protecting group,

LG is a suitable leaving group and R⁹ is H or SO₂CH₃.

Preferably said process comprises deprotection steps to obtain a compound of formula (I).

Preferably said process comprises a step for isolating said compound of formula (I).

In a preferred embodiment, said process comprises the step of reacting said compound of formula (7) with a compound of formula (5)

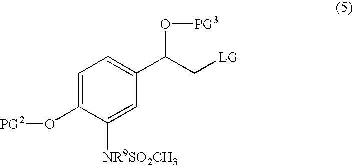

where R⁹ is H to obtain a compound of formula (3)

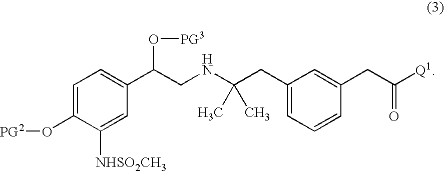

Preferably, said compound of formula (3) is then deprotected to obtain a compound of formula (I).

Preferably, two deprotection steps are carried out to remove PG² and PG³ and obtain a compound of formula (I).

Preferably, a first deprotection step is carried out to remove PG³ to obtain a compound of formula (2)

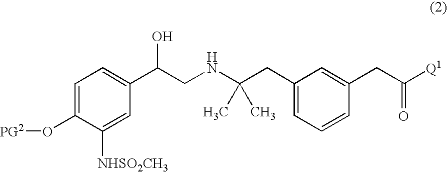

or a salt thereof.

Preferably said compound of formula (3) is not isolated and the first deprotection step is carried out directly.

Preferably a salt of compound of formula (2) is prepared and used in the next step. A preferred salt of compound of formula (2) is the dibenzoyl-(L)-tartrate salt.

Preferably a second deprotection step is carried out to remove PG² and obtain a compound of formula (I).

In another preferred embodiment, said compound of formula (7) is reacted with a compound of formula (5)

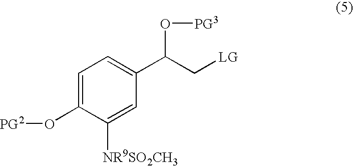

wherein R⁹ is SO₂CH₃ to obtain a compound of formula (3a)

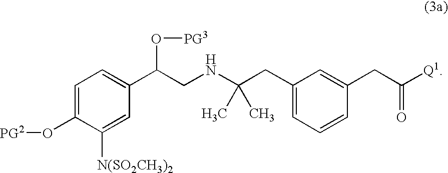

Preferably, said compound of formula (3a) is then deprotected to obtain a compound of formula (I).

Preferably, three deprotection steps are carried out to remove a SO₂CH₃ group, PG² and PG³.

Preferably, a first deprotection step is carried out to remove PG³ and obtain a compound of formula (4)

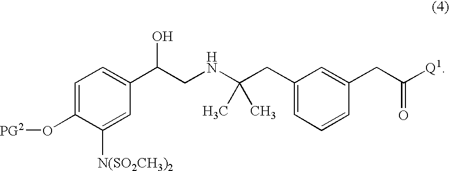

Preferably, a second deprotection step is carried out to remove a SO₂CH₃ group and obtain a compound of formula (2)

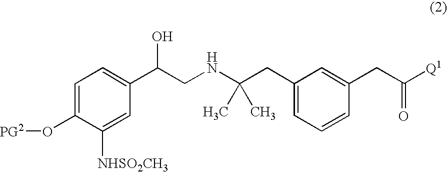

or a salt thereof.

Preferably a third deprotection step is carried out to remove PG² and obtain a compound of formula (I).

In another preferred embodiment, said compound of formula (7) is reacted with a compound of formula (6)

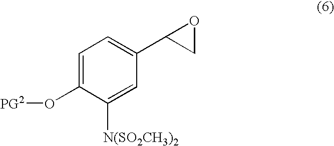

where PG² is a suitable phenol protecting group, to obtain a compound of formula

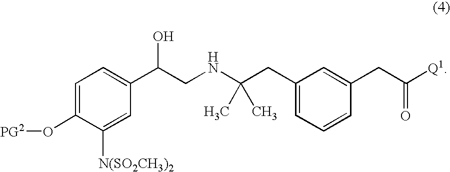

Preferably, said compound of formula (4) is then deprotected to obtain a compound of formula (I).

Preferably, two deprotection steps are carried out to remove SO₂CH₃ and PG² and obtain a compound of formula (I).

Preferably, a first deprotection step is carried to remove a SO₂CH₃ group and obtain a compound of formula (2)

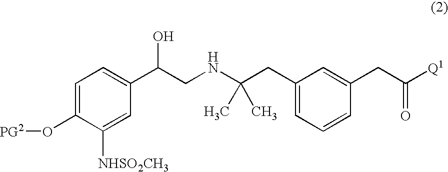

or a salt thereof.

Preferably a second deprotection step is carried out to remove PG² and obtain a compound of formula (I).

Preferably, LG is bromide.

Preferably, PG³ is TBDMS.

Preferably, PG² is benzyl.

In preferred embodiment, said compound of formula (7) is prepared by reacting a compound of formula (10)

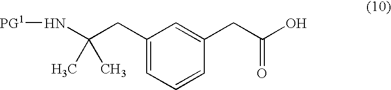

wherein PG¹ is a suitable amino protecting group, with Q¹-H or as salt thereof, wherein Q¹ is as defined above, to obtain a compound of formula (8)

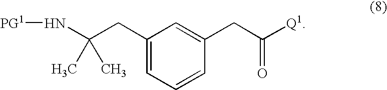

Preferably, a deprotection step is carried out to remove PG¹ and obtain said compound of formula (7).

Preferably, said compound of formula (10) is prepared by hydrolysis of a compound of formula (11)

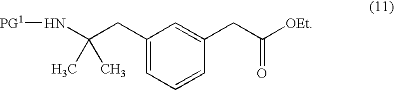

Preferably, said compound of formula (11) is prepared by protection of a compound of formula (12),

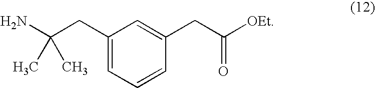

Preferably, PG¹ is Boc, trichloroacetyl or chloroacetyl.

In another preferred embodiment, said compound of formula (8) is prepared by reacting a compound of formula (19)

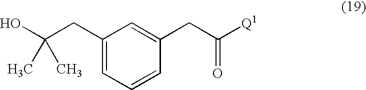

with an alkyl nitrile or an aryl nitrile, preferably trichloroacetonitrile or chloroacetonitrile.

Preferably, said compound of formula (19) is prepared by reacting a compound of formula (15).

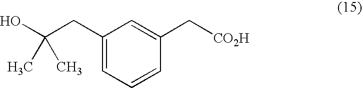

with Q¹-H or a salt thereof, wherein Q¹ is as defined above.

Compound of formula (16), which is a precursor of compound of formula (12) may be prepared by hydrolysis in the presence of an enzyme.

In a preferred embodiment, the compound of formula (16)

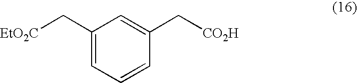

is prepared by hydrolyzing a compound of formula (18)

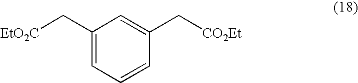

in the presence of an enzyme selected from a lipase, an esterase or a protease.

Preferably, said enzyme is selected from Mucor Miehei esterase, Rhizomucor Miehei lipase, Thermomuces Languinosus lipase, Penicillin acylase.

More preferably, said enzyme is Thermomuces Languinosus lipase.

Preferably, the hydrolysis of said compound of formula (18) is carried out at a pH between 5 and 9 and a temperature between 10° C. and 40° C. in water, in the presence of a suitable buffering agent, and optionally in the presence of a suitable base.

The present invention also relates to the intermediates used in said process of the invention.

In a preferred embodiment, the invention relates to the following intermediates:

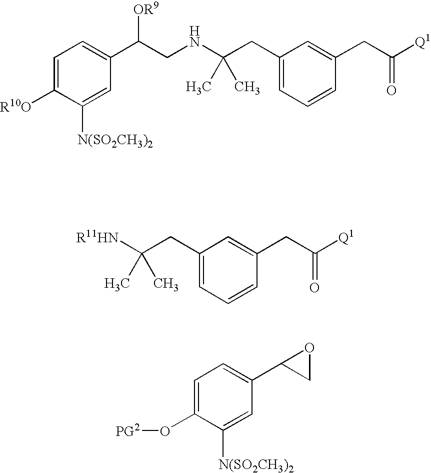

wherein Q¹ is as defined above, R¹⁰ is H or PG² where PG² is a suitable phenol protecting group, R⁹ is H or PG³ where PG³ is a suitable hydroxyl protecting group, and R¹¹ is H, PG¹ where PG¹ is a suitable amino protecting group.

Preferred intermediates are:
2-(3-{2-[((2R)-2-{4-Benzyloxy-3-[(dimethylsulfonyl)amino]phenyl}-2-{[tert-butyl(dimethyl)silyl]oxy}ethyl)amino]-2-methylpropyl}phenyl)-N-[(4′-hydroxybiphenyl-3-yl)methyl]-acetamide;
2-(3-{2-[(((2R)-2-{4-Benzyloxy-3-[(dimethylsulfonyl)amino]phenyl}-2-hydroxyethyl)amino]-2-methylpropyl}phenyl)-N-[(4′-hydroxybiphenyl-3-yl)methyl]-acetamide;
tert-Butyl-[2-(3-{[(4′-hydroxybiphenyl-3-ylmethyl)-carbamoyl]-methyl}-phenyl)-1,1-(dimethyl)ethyl]carbamate;
2,2,2-Trichloro-N-[2-(3-{[4′-hydroxybiphenyl-3-ylmethyl)-carbamoyl]-methyl}-phenyl)-1,1-dimethyl-ethyl]acetamide,
2-Chloro-N-{2-[3-(2-{[(4′-hydroxybiphenyl-3-yl)methyl]amino}-2-oxoethyl)phenyl]-1,1-dimethylethyl}acetamide,
2-[3-(2-Amino-2-methylpropyl)-phenyl]-N-[(4′-hydroxybiphenyl-3-yl)methyl]acetamide, and, N—[(R)-2-benzyloxy-5-oxiranyl-phenyl]-dimethanesulfonamide.

In the here above general formula (I), C₁-C₄ alkyl denote a straight-chain or branched group containing 1, 2, 3 or 4 carbon atoms. This also applies if they carry substituents or occur as substituents of other radicals, for example in O—(C₁-C₄)alkyl radicals, S—(C₁-C₄)alkyl radicals etc . . . . Examples of suitable (C₁-C₄)alkyl radicals are methyl, ethyl, n-propyl, iso-propyl, n-butyl, iso-butyl, sec-butyl, tert-butyl . . . . Examples of suitable (C₁-C₄)alkoxy radicals are methoxy, ethoxy, n-propyloxy, iso-propyloxy, n-butyloxy, iso-butyloxy, sec-butyloxy and tert-butyloxy . . . .

Halo denotes a halogen atom selected from the group consisting of fluoro, chloro, bromo and iodo in particular fluoro or chloro.

The term C₃-C₇ cycloalkyl includes cyclopropyl, cyclobutyl, cyclopentyl, cyclohexyl and cycloheptyl.

A suitable hydroxyl-protecting group includes tert-butyl(dimethyl)silyl (TBDMS), triethylsilyl, tert-butyl(diphenyl)silyl, tri(isopropyl)silyl, tetrahydropyranyl, methoxymethyl, benzyloxymethyl, 1-ethoxyethyl and benzyl. A preferred hydroxyl-protecting group is tert-butyl(dimethyl)silyl or triethylsilyl.

A suitable phenol-protecting group includes benzyl, methyl, methoxymethyl, benzyloxymethyl, TBDMS, 4-methoxybenzyl and 4-chlorobenzyl. A preferred phenol-protecting group is benzyl. A suitable amino protecting group includes tert-butoxycarbonyl (Boc), chloroacetyl, trichloroacetyl, acetyl, trifluoroacetyl, benzyloxycarbonyl, formyl, phenylacyl, allyloxycarbonyl, 2-(trimethylsilyl)ethoxycarbonyl or 2,2,2-trichloroethoxycarbonyl. A preferred amino protecting group is Boc, chloroacetyl or trichloroacetyl.

A suitable leaving group includes bromide, 4-bromobenzenesulfonyl, chloride, iodide, methanesulfonyl, 4-nitrobenzenesulfonyl, p-toluenesulfonyl and trifluoromethanesulfonyl. A preferred leaving group is bromide, chloride or p-toluenesulfonyl.

In the compounds of formula (I) and in the intermediates for their preparation, Q¹ is preferably

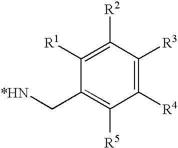

Preferably, R¹, R², R³, R⁴ and R⁵ are the same or different and are selected from H, C₁-C₄ alkyl, OR⁵, SR⁶, halo, preferably chloro, CF₃, OCF₃, SO₂NR⁷R⁸, CONR⁷R⁸, NR⁷R⁸, NHCOR⁷, provided at least 2 of R¹ to R⁵ are H;

wherein R⁷ and R⁸ are the same or different and are selected from H or C₁-C₄ alkyl.

Preferably R¹, R², R³, R⁴ and R⁵ are the same or different and are selected from H, OH, CH₃, OCH₂—CH₃, SCH₃, halo, preferably chloro, CF₃, OCF₃, provided at least 2 of R¹ to R⁵ are H.

Preferably R¹, R², R³, R⁴ and R⁵ are the same or different and are selected from H or halo, preferably chloro provided at least 2 of R¹ to R⁵ are H.

Preferably, R² and R³ are chloro and R¹, R⁴ and R⁵ are H.

Preferably, one of R¹ to R⁵ is OH.

Preferably, one of R¹, R², R³, R⁴ and R⁵ is phenyl substituted by OH and the others are H.

Preferably, R² is 4-hydroxy-phenyl and R¹, R³, R⁴ and R⁵ are H.

Preferably, the process of the invention is used for the preparation of the following compounds:
N-[(4′-hydroxybiphenyl-4-yl)methyl]-2-(3-{2-[((2R)-2-hydroxy-2-{4-hydroxy-3-[(methylsulfonyl)amino]phenyl}ethyl)amino]-2-methylpropyl}phenyl)acetamide;
N-(4-chloro-2-hydroxybenzyl)-2-(3-{2-[((2R)-2-hydroxy-2-{4-hydroxy-3-[(methylsulfonyl)amino]phenyl}ethyl)amino]-2-methylpropyl}phenyl)acetamide;
N-[(4′-hydroxybiphenyl-3-yl)methyl]-2-(3-{2-[((2R)-2-hydroxy-2-{4-hydroxy-3-[(methylsulfonyl)amino]phenyl}ethyl)amino]-2-methylpropyl}phenyl)acetamide;
N-(3,4-dichlorobenzyl)-2-(3{-2-[((2R)-2-hydroxy-2-{4-hydroxy-3-[(methylsulfonyl)amino]phenyl}ethyl)amino]-2-methylpropyl}phenyl)acetamide;
2-(3-{2-[((2R)-2-hydroxy-2-{4-hydroxy-3-[(methylsulfonyl)amino]phenyl}ethyl)amino]-2-methyl propyl}phenyl)-N-[(6-hydroxy-2-naphthyl)methyl]acetamide;
2-(3-{2-[((2R)-2-hydroxy-2-{4-hydroxy-3-[(methylsulfonyl)amino]phenyl}ethyl)amino]-2-methyl propyl}phenyl)-N-[(2-hydroxy-1-naphthyl)methyl]acetamide, and,
2-(3-{2-[((2R)-2-hydroxy-2-{4-hydroxy-3-[(methylsulfonyl)amino]phenyl}ethyl)amino]-2-methyl propyl}phenyl)-N-[3-hydroxy-5-(trifluoromethyl)benzyl]acetamide.

In a preferred embodiment the invention relates to a process for the preparation of a compound of formula (I) where the carbon atom substituted with a hydroxyl group is in the R configuration:

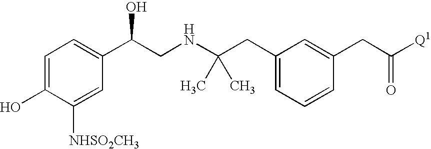

wherein Q¹ is as defined above and intermediates for its preparation.

In a preferred embodiment, the invention relates to a process for the preparation of a compound of formula (Ia):

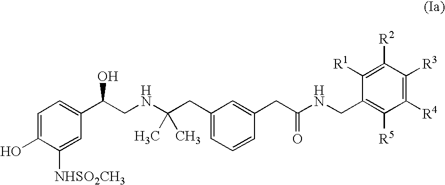

wherein R¹ to R⁵ are as defined above and intermediates for its preparation.

The process of the invention is illustrated by the below schemes:

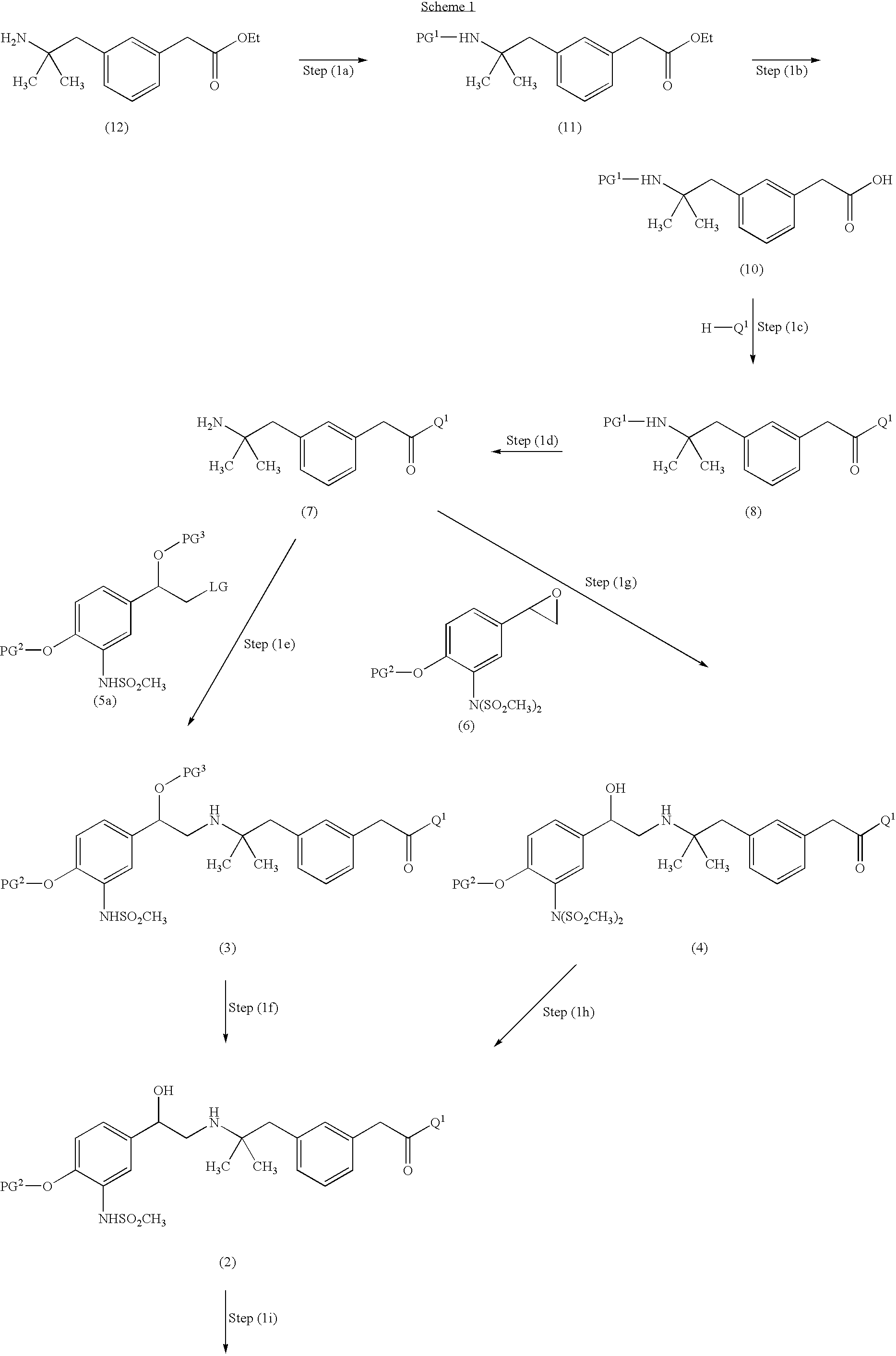

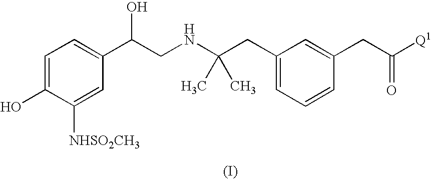

Q¹ is as defined above.

PG¹ is a suitable amino protecting group. Preferably, PG¹ is Boc, chloroacetyl or trichloroacetyl.

PG² is a suitable phenol protecting group. Preferably, PG² is benzyl.

PG³ is a suitable hydroxyl-protecting group. Preferably, PG³ is TBDMS.

LG is a suitable leaving group. Preferably, LG is bromide.

Preferably, in the above scheme, the carbon atom substituted with a hydroxyl or an OPG³ group is in the R configuration.

Q¹-H is selected from

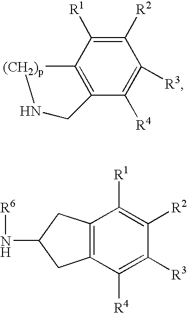

and HNR⁶-Q²-A wherein p, Q², A, R¹ to R⁵ and R⁶ are as defined above.

In step (1a), the amine of formula (12) is reacted with a protecting agent such as di-tert-butyl dicarbonate or benzyl chloroformate in the presence of an amine such as 4-dimethylaminopyridine or triethylamine in a suitable solvent such as tetrahydrofuran (THF). Other suitable protecting agents are described in the textbook “Protective Groups in Organic Synthesis” by T. W. Greene and P. G. M. Wuts. Typical conditions comprise of 1.0 equivalent of compound (12), 1 to 3 equivalents of di-tert-butyl dicarbonate and 0.05 to 2 equivalents of 4-dimethylaminopyridine in a suitable solvent such as tetrahydrofuran at 10 to 50° C. for 12 to 48 hours.

In step (1b) an ester of formula (11) is hydrolyzed to a carboxylic acid of formula (10) using standard methodology as described in the textbook “Protective Groups in Organic Synthesis” by T. W. Greene and P. G. M. Wuts. Typical conditions comprise of 1.0 equivalent of compound (11) and 2 to 5 equivalents of sodium hydroxide in a suitable solvent such as a mixture of water and tetrahydrofuran or ethanol at 10 to 50° C. for 12 to 48 hours.

In step (1c) a carboxylic acid of formula (10) is reacted with a primary or secondary amine (or a salt thereof) of formula H-Q¹ in the presence of a suitable base such as triethylamine or diisopropylethylamine and a suitable coupling reagent such as 1-(3-dimethylaminopropyl)-3-ethylcarbodiimide hydrochloride, dicyclohexylcarbodiimide, carbonyl diimidazole, pivaloyl chloride or isobutyl chloroformate, optionally in the presence of a suitable additive such as 1-hydroxybenzotriazole or N-hydroxysuccinimide in a suitable solvent such as dimethylformamide, propionitrile, acetonitrile or pyridine. Typical conditions comprise 1.0 equivalent of compound (10), 1.0 to 1.5 equivalents of compound of formula H-Q¹, 1 to 5 equivalents of base and 1.05 to 2 equivalents of 1-(3-dimethylaminopropyl)-3-ethylcarbodiimide hydrochloride in a suitable solvent such as propionitrile, dimethylformamide or acetonitrile at 10 to 40° C. for 1 to 24 hours.

In step (1d), PG¹ may be removed using standard methodology as described in “Protective Groups in Organic Synthesis” by T. W. Greene and P. G. M. Wuts. When PG¹ is tert-butoxycarbonyl, typical conditions comprise 1.0 equivalent of compound (8) and 1 to 10 equivalents of hydrochloric acid or trifluoroacetic acid, in a suitable solvent such as dichloromethane or a mixture of ethanol and 1,4-dioxane at 10 to 50° C. for 12 to 100 hours.

In step (1e) an amine of formula (7) is reacted with an activated compound of formula (5a) optionally in the presence of a base such as sodium hydrogen carbonate, triethanolamine, dipotassium hydrogenphosphate or diisopropylethylamine in the presence of a suitable solvent such as propionitrile, butyronitrile, 1-methyl-2-pyrrolidinone, n-propyl acetate, n-butyl acetate or 4-methyl-2-pentanone, at a temperature between 50° C. and 150° C. for 12 to 48 hours. Typical conditions comprise of 1.0 equivalent of compound (7), 0.5 to 2.0 equivalents of compound (5a) and 2 to 5 equivalents of sodium hydrogen carbonate in butyronitrile or n-butyl acetate at 110 to 120° C. for 24 to 48 hours.

In step (1f), PG³ may be removed using standard methodology as described in “Protective Groups in Organic Synthesis” by T. W. Greene and P. G. M. Wuts. When PG³ is tert-butyldimethylsilyl, a deprotecting agent such as tetrabutylammonium fluoride, HF, or triethylamine trihydrofluoride in the presence of a suitable solvent such as tetrahydrofuran, ethanol, methanol or propionitrile may be used. Typical conditions comprise of 1.0 equivalent of compound (3), and 1-5 equivalents of triethylamine trihydrofluoride, in a suitable solvent such as methanol, tetrahydrofuran, a mixture of butyronitrile and methanol, or a mixture of n-butyl acetate, ethyl acetate and methanol, at 25 to 40° C. for 1 to 24 hours.

In step (1 g), an amine of formula (7) is reacted with an epoxide of formula (6) in a suitable solvent such as propionitrile, butyronitrile or n-butanol, at a temperature between 80° C. and 150° C. for 12 to 60 hours. Typical conditions comprise of 1.0 equivalents of compound (7) and 0.5 to 2 equivalents of compound (6) in a suitable solvent such as butyronitrile or n-butanol at 100 to 130° C. for 12 to 48 hours.

In step (1h), a compound of formula (4) is treated with a suitable deprotecting reagent such as sodium hydroxide, potassium hydroxide, tetrabutylammonium fluoride or potassium carbonate in the presence of a suitable solvent such as tetrahydrofuran or a mixture of water and a water-miscible alcohol such as ethanol or methanol, at 10 to 50° C. for 3 to 100 hours. Typical conditions comprise of 1.0 equivalent of compound (4) and 4-10 equivalents of sodium hydroxide in a mixture of ethanol and water at 25 to 40° C. for 12 to 100 hours.

In step 1(i), PG² may be removed using standard methodology as described in “Protective Groups in Organic Synthesis” by T. W. Greene and P. Wutz. When PG² is benzyl, typical conditions comprise of 1.0 equivalent of compound (2), in the presence of a suitable catalyst such as 20% Pd(OH)₂/C or 5% Pd/C, in a suitable solvent such as ethanol, aqueous ethanol, tetrahydrofuran, aqueous tetrahydrofuran, ethylene glycol, propylene glycol or dimethylformamide, under 40 to 80 psi of hydrogen, at 25 to 60° C. for 2 to 54 hours.

Alternatively, the deprotection step (1i) may be carried out before the deprotection step (1f), as illustrated in the Scheme below.

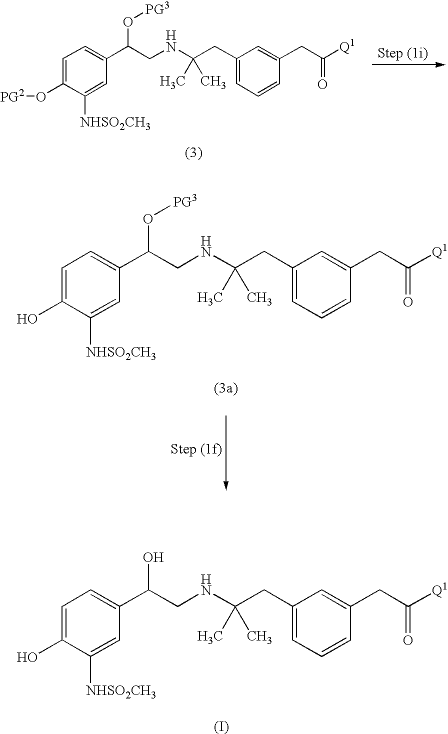

In this embodiment, both PG² and PG³ may be removed using standard methodology as described in “Protective Groups in Organic Synthesis” by T. W. Greene and P. Wutz. When PG² is benzyl, typical conditions for step (1i) comprise 1.0 equivalent of compound (3), in the presence of a suitable catalyst such as 20% Pd(OH)₂/C or 5% Pd/C, in a suitable solvent such as ethanol, tetrahydrofuran, ethyl acetate or a mixture of ethyl acetate and n-butyl acetate, under 40 to 80 psi of hydrogen, at 25 to 60° C. for 2 to 48 hours. When PG³ is tert-butyldimethylsilyl, typical conditions for step (1f) comprise 1.0 equivalent of compound (3a) and 1.0 to 10.0 equivalents of ammonium fluoride in a suitable solvent such as aqueous methanol, aqueous ethanol or aqueous acetonitrile at 10 to 40° C. for 1 to 48 hours.

Preferably, in the above compounds, the carbon atom substituted with an hydroxyl or an OPG³ group is in the R configuration.

Alternatively, the deprotection step (1i) may be carried out before the deprotection step (1 h).

Alternatively, step (1e) may be replaced by the below steps, using a compound of formula (5b).

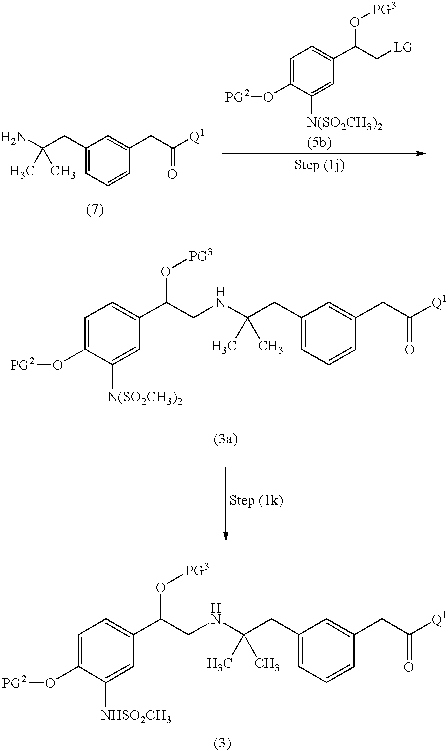

The conditions of steps (1j) and (1k) are identical to those disclosed for steps (1e) and (1h) above respectively. Preferably, in the above compounds, the carbon atom substituted with an hydroxyl or an OPG³ group is in the R configuration.

In step (1j) an amine of formula (7) is reacted with an activated compound of formula (5b) optionally in the presence of a base such as sodium hydrogen carbonate, triethanolamine, dipotassium hydrogenphosphate or diisopropylethylamine in the presence of a suitable solvent such as propionitrile, butyronitrile, 1-methyl-2-pyrrolidinone, n-propyl acetate, n-butyl acetate or 4-methyl-2-pentanone, at a temperature between 50° C. and 150° C. for 12-48 hours. Typical conditions comprise of 1.0 equivalent of compound (7), 0.5 to 2.0 equivalents of compound (5b) and 2 to 5 equivalents of sodium hydrogen carbonate in butyronitrile at 110 to 120° C. for 24 to 48 hours.

In step (1k), a compound of formula (3a) is treated with a suitable deprotecting reagent such as sodium hydroxide, potassium hydroxide, tetrabutylammonium fluoride or potassium carbonate in the presence of a suitable solvent such as tetrahydrofuran or a mixture of water and a water-miscible alcohol such as ethanol or methanol, at 10 to 50° C. for 3 to 100 hours. Typical conditions comprise of 1.0 equivalent of compound (3a) and 4 to 10 equivalents of sodium hydroxide in a mixture of ethanol and water at 25 to 40° C. for 12 to 100 hours.

Alternatively, the sequence of deprotection steps for the conversion of a compound of formula (3a) to a compound of formula (I) can be varied such that any of PG², PG³ and the methanesulfonamide can be removed in any order.

Compounds of formula (5) wherein PG² is benzyl, PG³ is TBDMS and LG is bromide may be prepared as disclosed in the following scheme:

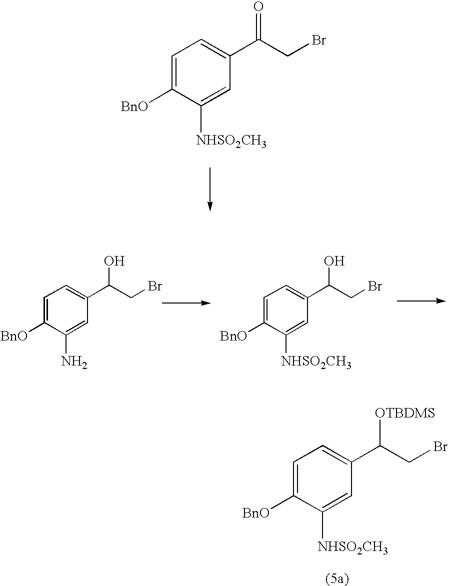

Details of the preparation of compound (5a) are disclosed in the examples.

Preferably, in the above compounds, the carbon atom substituted with an hydroxyl or an OTBDMS group is in the R configuration.

Compounds of formula (5b) and (6) may be prepared by the process according to Scheme 2

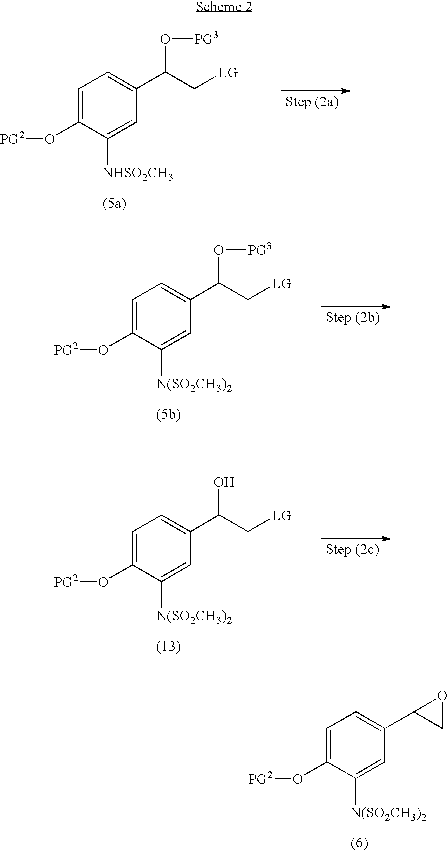

PG², PG³ and LG are as defined above.

Preferably, in the above compounds, the carbon atom substituted with a hydroxyl or an OPG³ group is in the R configuration.

The R isomer of compound of formula (6) is also preferred:

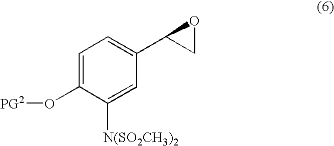

In step (2a), a compound of formula (5a) is treated with methanesulfonylchloride in the presence of a suitable base such as diisopropylethylamine, triethylamine, sodium hydride, lithium diisopropylamide or n-butyl lithium in a suitable solvent such as acetonitrile, propionitrile, tetrahydrofuran, dichloromethane, 1,4-dioxane or dimethylformamide at a temperature between −80° C. and 80° C. for 1 to 24 hours. Typical conditions comprise of 1.0 equivalent of compound (5a), 2-5 equivalents of diisopropylethylamine and 1 to 5 equivalents of methanesulfonyl chloride in a suitable solvent such as acetonitrile for 1 to 5 hours at 5 to 25° C.

In step (2b), PG³ may be removed using standard methodology as described in “Protective Groups in Organic Synthesis” by T. W. Greene and P. G. M. Wuts. When PG³ is tert-butyldimethylsilyl, a deprotecting agent such as tetrabutylammonium fluoride, HF, or triethylamine trihydrofluoride in the presence of a suitable solvent such as tetrahydrofuran, methanol, ethanol or propionitrile may be used. Typical conditions comprise of 1.0 equivalent of compound (5b), and 1 to 5 equivalents of triethylamine trihydrofluoride, in a suitable solvent such as methanol, or tetrahydrofuran, at 25 to 40° C. for 12 to 48 hours.

In step (2c), a compound of formula (13) is reacted with a suitable base such as potassium carbonate, triethylamine, sodium hydride, sodium carbonate, diisopropylethylamine in the presence of a suitable solvent such as tetrahydrofuran, methanol, ethanol, dichloromethane, water for 2 to 24 hours at 10-40° C. Typical conditions comprise of 1.0 equivalent of compound (13) and 1 to 5 equivalents of potassium carbonate in a suitable solvent such as a mixture of methanol and tetrahydrofuran at 20 to 25° C. for 12 to 18 hours.

Compounds of formula H-Q¹ are either commercially available or may be prepared by conventional methods well known to the one skilled in the art (e.g. reduction, oxidation, alkylation, transition metal-mediated coupling, protection, deprotection etc . . . . ) from commercially available material. Examples of such preparations are disclosed in WO2004/032921, WO2004/108676, WO2004/108675 and WO2004/100950.

The compound of formula (12) may be prepared according to the process of the following scheme 3:

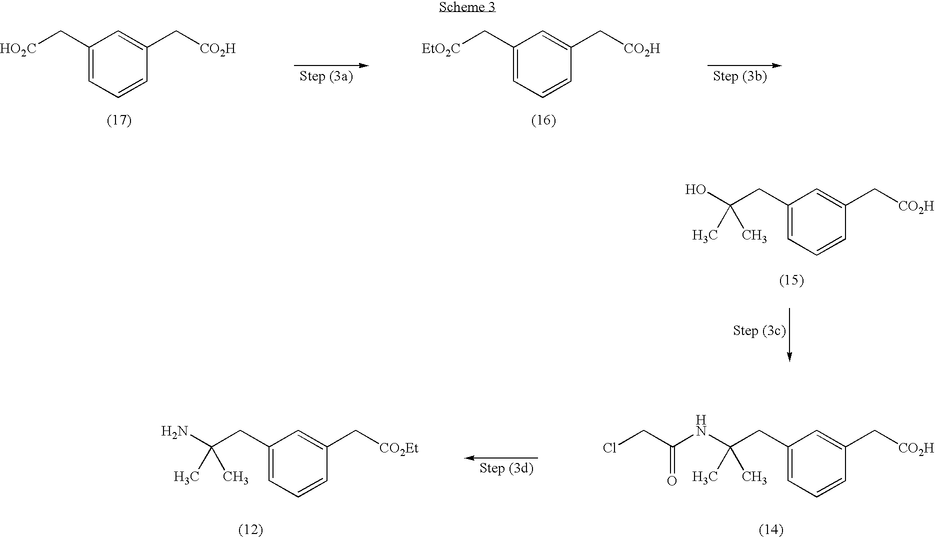

Details of the preparation of the compound of formula (12) are disclosed in the examples.

Alternatively, step (3a) may be replaced by the following steps:

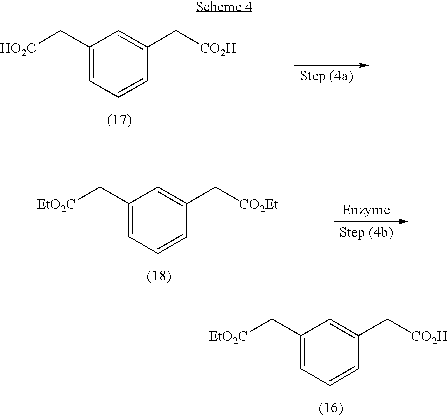

In step (4a), the diester of formula (18) is prepared by esterification of the diacid of formula (17) according to any method well-known to the one skilled in the art to prepare an ester from an acid without modifying the rest of the molecule. Typical conditions comprise of 1.0 equivalent of the diacid of formula (17) reacting with an alcoholic solvent, preferably ethanol, in the presence of an acid catalyst such as hydrogen chloride or sulfuric acid at a temperature between 10° C. and 100° C. for 6 to 24 hours.

In step (4b), the diester of formula (18) is selectively hydrolyzed to the monoester of formula (16) in the presence of a suitable enzyme known in the art, such as a lipase, esterase or protease, preferably a lipase. Preferred enzymes are Mucor Miehei esterase, Rhizomucor Miehei lipase, Thermomuces Languinosus lipase, Penicillin acylase. More preferably, the reaction is carried out with Lipolase® (Thermomuces Languinosus lipase, (EC No 3.1.1.3)) at a pH between 5 and 9 and a temperature between 10° C. and 40° C. in water in the presence of a suitable buffering agent such as calcium acetate, dipotassium hydrogenphosphate or triethanolamine, and optionally in the presence of a suitable base such as sodium hydroxide, potassium hydroxide or lithium hydroxide. Typical conditions comprise 1.0 equivalent of the diester of formula (18) reacting with 5 to 200 ml of Lipolase® (liquid formulation) in a calcium acetate buffer solution at a temperature between 20° C. and 40° C., maintaining the pH between 5.5 and 6.8 by the addition of a base such as sodium hydroxide or potassium hydroxide for 12 to 24 hours.

Alternatively, step (3d) may be replaced by the following steps, as illustrated in Scheme 5:

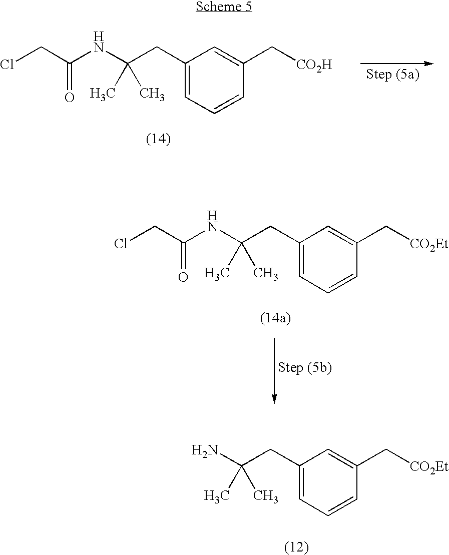

In step (5a), the ester of formula (14a) is prepared by esterification of the acid of formula (14) according to any method well-known to the one skilled in the art to prepare an ester from an acid without modifying the rest of the molecule. Typical conditions comprise of 1.0 equivalent of the acid of formula (14) reacting with an alcoholic solvent, preferably ethanol, in the presence of an acid catalyst such as hydrogen chloride or sulfuric acid at a temperature between 20° C. and 100° C. for 1 to 12 hours.

In step (5b), the amide of formula (14a) is deprotected using standard using standard methodology as described in “Protective Groups in Organic Synthesis” by T. W. Greene and P. G. M. Wuts. Typical conditions comprise of 1.0 equivalent of the chloroacetamide of formula (14a) reacting with 1 to 3 equivalents of thiourea in a suitable solvent such as a mixture of ethanol and acetic acid at a temperature between 50° C. and 120° C. for 12 to 24 hours.

Alternatively, compounds of formula (7) may be prepared according to the following scheme 6:

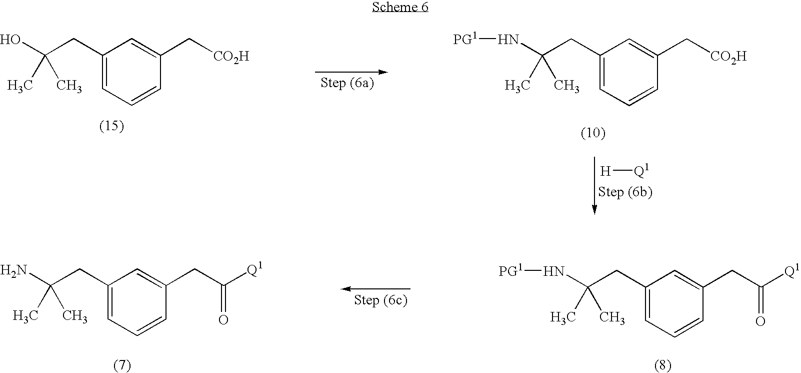

In scheme 6, PG¹ is preferably trichloroacetyl or chloroacetyl. More preferably, PG¹ is trichloroacetyl.

In step (6a), the tertiary alcohol of formula (15) is treated with an alkyl or aryl nitrile and an acid catalyst to give the amide of formula (10). Preferably, the tertiary alcohol of formula (15) is reacted with trichloroacetonitrile or chloroacetonitrile in the presence of an acid such as sulfuric acid, acetic acid, trifluoroacetic acid to give the protected amide of formula (20). Typical conditions comprise the addition of between 1 and 3 mL of concentrated (98%) sulfuric acid per gram of alcohol of formula (1-2 equivalents of trichloroacetonitrile in a suitable solvent such as acetic acid at a temperature between 0° C. and 25° C. for 1 to 8 hours.

In step (6b), the carboxylic acid of formula (10) is reacted with a primary or secondary amine of formula H-Q¹ or a salt thereof in the presence of a suitable base such as triethylamine or diisopropylethylamine and a suitable coupling reagent such as 1-(3-dimethylaminopropyl)-3-ethylcarbodiimide hydrochloride, dicyclohexylcarbodiimide, carbonyl diimidazole, pivaloyl chloride or isobutyl chloroformate, optionally in the presence of a suitable additive such as 1-hydroxybenzotriazole or N-hydroxysuccinimide in a suitable solvent such as ethyl acetate, dimethylformamide, propionitrile, acetonitrile or pyridine. Typical conditions comprise 1.0 equivalent of compound of formula (10), 0.8 to 1.2 equivalents of compound of formula H-Q¹, 1 to 5 equivalents of base, 1 to 2 equivalents of 1-hydroxybenzotriazole and 1.05 to 2 equivalents of 1-(3-dimethylaminopropyl)-3-ethylcarbodiimide hydrochloride in a suitable solvent such as ethyl acetate, propionitrile, dimethylformamide at 20 to 60° C. for 12 to 36 hours.

In step (6c), PG¹ is removed using standard methodology as described in “Protective Groups in Organic Synthesis” by T. W. Greene and P. G. M. Wuts or other methods well-known to those experienced in the art. When PG¹ is trichloroacetyl, typical conditions comprise 1.0 equivalent of compound (8) and 2 to 10 equivalents of a suitable base such as potassium hydroxide or sodium hydroxide in a suitable solvent such as water, ethanol or methanol or preferably a mixture of water and ethanol at a temperature between 30° C. and 80° C. for 16 to 36 hours.

Alternatively, compounds of formula (7) may be prepared according to the following scheme 7:

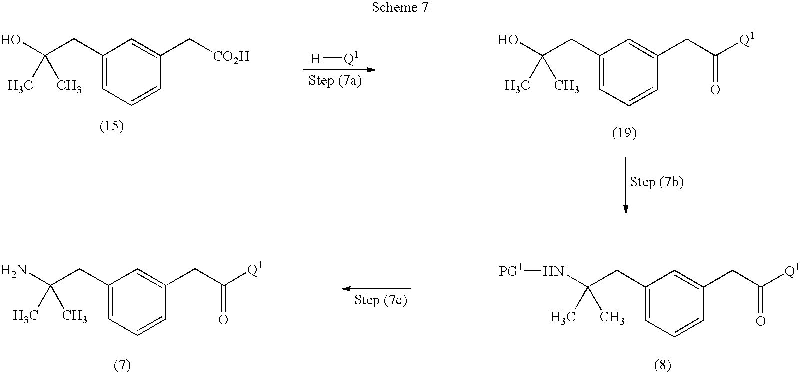

In scheme 7, PG¹ is preferably trichloroacetyl or chloroacetyl. More preferably, PG¹ is chloroacetyl.

In step (7a), the carboxylic acid of formula (15) is reacted with a primary or secondary amine of formula H-Q¹ or a salt thereof in the presence of a suitable base such as triethylamine or diisopropylethylamine and a suitable coupling reagent such as 1-(3-dimethylaminopropyl)-3-ethylcarbodiimide hydrochloride, dicyclohexylcarbodiimide, carbonyl diimidazole, pivaloyl chloride or isobutyl chloroformate, optionally in the presence of a suitable additive such as 1-hydroxybenzotriazole or N-hydroxysuccinimide in a suitable solvent such as dichloromethane, ethyl acetate, dimethylformamide, propionitrile, acetonitrile or pyridine. Typical conditions comprise 1.0 equivalent of compound of formula (15), 0.8 to 1.2 equivalents of compound of formula H-Q¹, 1 to 5 equivalents of base, 0.4 to 2 equivalents of 1-hydroxybenzotriazole and 1 to 2 equivalents of 1-(3-dimethylaminopropyl)-3-ethylcarbodiimide hydrochloride in a suitable solvent such as dichloromethane, ethyl acetate, propionitrile, dimethylformamide at 20 to 60° C. for 1 to 24 hours.

In step (7b), the tertiary alcohol of formula (19) is treated with an alkyl or aryl nitrile and an acid catalyst to give the amide of formula (8). Preferably, the tertiary alcohol of formula (19) is reacted with trichloroacetonitrile or chloroacetonitrile in the presence of an acid such as sulfuric acid, acetic acid, trifluoroacetic acid to give the protected amide of formula (8). Typical conditions comprise the addition of between 2 and 5 mL of trifluoroacetic acid per gram of alcohol of formula (19) to a solution of 1.0 equivalent of the alcohol of formula (19) and 2 to 5 mL of chloroacetonitrile per gram of alcohol of formula (19) at a temperature between 0° C. and 75° C. for 1 to 8 hours. Compound of formula (8) can be isolated before carrying out step (7c).

In step (7c), PG¹ is removed using standard methodology as described in “Protective Groups in Organic Synthesis” by T. W. Greene and P. G. M. Wuts or other methods well-known to those experienced in the art. When PG¹ is chloroacetyl, typical conditions comprise 1.0 equivalent of compound (8) and 2 to 8 equivalents of thiourea in a suitable solvent such as acetic acid, isopropanol, ethyl acetate, isopropyl acetate, preferably acetic acid at a temperature between 50° C. and 120° C. for 1 to 36 hours.

The process of the invention is illustrated by the below examples.

### EXAMPLE 1

### Preparation of N-(4′-Hydroxy-biphenyl-3-ylmethyl)-2-(3-{2-[2-hydroxy-2-(4-hydroxy-3-methyl-phenyl)-ethylamino]-2-methyl-propyl}-phenyl)-acetamide

### Preparation 1

### Diethyl 2,2′-(1,3-phenylene)diacetate

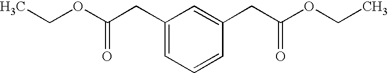

To a suspension of 2,2′-(1,3-phenylene)diacetic acid (45.55 Kg; 234.6 moles) in ethanol (455.5 L) was charged concentrated sulfuric acid (1.82 L). The resulting thin suspension was heated to reflux for 20 hours. The reaction was cooled to room temperature and ethanol was removed at atmospheric pressure and replaced with toluene (136.5 L). The toluene solution was washed with 5% aqueous sodium hydrogen carbonate (1×91 L), and then concentrated to an approximately 1 mL/g solution in toluene and taken on to the next step. Analysis of an aliquot concentrated to dryness under vacuum indicated ˜100% yield.

¹H NMR (CD₃OD, 400 MHz) δ: 1.21 (t, 6H), 3.59 (s, 4H), 4.10 (q, 4H), 7.15-7.27 (m, 4H) ppm.

MS (electrospray): m/z 251 [M+H]⁺

### Preparation 2

### [3-(2-Oxo-propyl)-phenyl]-acetic acid ethyl ester

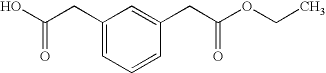

Lipolase® (Thermomyces lanuginosus lipase solution; 9.4 L) was added to a 0.2M solution of calcium acetate in water (117.5 L) and the homogeneous solution was stirred at ambient temperature for 30 minutes. The toluene solution of the product from preparation 1 (29.35 Kg, 117.3 moles) was added and the reaction was stirred at ambient temperature. The pH was checked every 15 minutes and was maintained between 5.5 and 6.8 by addition of aliquots of a 1M aqueous sodium hydroxide solution. The reaction was complete after 48 h. The pH was adjusted to 3-4 using 1M aqueous hydrochloric acid and ethyl acetate was added (117 L). The biphasic mixture was filtered through a Gauthier filter to remove denatured enzyme. The mixture was then separated and the aqueous layer was extracted with ethyl acetate (2×117 L). The combined organic layers were extracted with saturated aqueous sodium hydrogen carbonate (3×149.69 L). The combined sodium hydrogen carbonate extracts were adjusted to pH 2 using 2M aqueous hydrochloric acid and the resulting solution was extracted with toluene (2×147 L). The toluene extract was then concentrated an approximately 1 mL/g toluene solution for use in the next step. Analysis of an aliquot concentrated to dryness under vacuum to give the title compound indicated a yield of 19.68 Kg; 75.6%.

¹H NMR (CD₃OD, 400 MHz) δ: 1.25 (t, 3H), 3.60 (m, 2H), 3.63 (m, 2H), 4.15 (q, 2H), 7.18-7.32 (m, 4H) ppm.

MS (electrospray): m/z 245 [M+Na]⁺

### Alternative Process for the Preparation of the Product of Preparation 2

To a suspension of 2,2′-(1,3-phenylene)diacetic acid (300.0 g; 1.54 moles) in THF (3.0 L) was charged absolute ethanol (85.41 g; 1.85 moles) and 37% aqueous hydrochloric acid (30 mL) which gave almost complete dissolution. The resulting thin suspension was heated to 50° C. until the reaction was complete (monitored by HPLC). Once the reaction was complete, the solvent was stripped and replaced with toluene (1.5 L) and the resulting suspension was stirred vigorously for 15 minutes before filtering under vacuum. The precipitate was washed with fresh toluene (300 mL) and then discarded (the precipitate is the starting 2,2′-(1,3-phenylene)diacetic acid). The toluene solution was extracted with saturated aqueous sodium hydrogen carbonate (1.35 L+2×300 mL). The combined sodium hydrogen carbonate extracts were adjusted to pH 5-6 using a combination of 37% hydrochloric acid and 2M hydrochloric acid and the resulting slightly milky solution was extracted with tert-butyl methyl ether (1.2 L+2×600 mL). The combined tert-butyl methyl ether extracts were washed with demineralised water (600 mL), dried over MgSO₄ and concentrated to dryness under vacuum to give the title compound as a pale straw-coloured oil (134.1 g).

### Preparation 3

### [3-(2-Hydroxy-2-methyl-propyl)-phenyl]-acetic acid

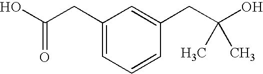

The toluene solution of the product of preparation 2 (3.59 Kg, solvent corrected; 16.15 moles) was dissolved in anhydrous tetrahydrofuran under nitrogen and cooled to 0-5° C. Methyl magnesium bromide (56.53 L of a 1M solution in tetrahydrofuran, 56.53 mol) was added to this solution at such a rate so as to maintain the temperature below 15° C. On completion of addition, the reaction was warmed to ambient temperature and stirred until complete. The reaction mixture was then cooled to between 0.0 and 5° C. and demineralised water (17.95 L) was added, maintaining the temperature below 15° C. Once the addition was complete, the pH was adjusted to between 1 and 2.5 by the addition of 5M hydrochloric acid. The mixture was extracted with isopropyl acetate (2×17.95 L), the combined organic extracts were washed with water (3×17.95 L) and then the isopropyl acetate was distilled and replaced with toluene, until a concentration of approximately 5 mL/g of toluene was achieved. The toluene solution was cooled to 5° C. and the resulting slurry was granulated for 2 h. The product was isolated by filtration, washing with toluene (3.59 L) to give the title compound as an off-white solid (2.29 Kg, 68%).

¹H NMR (CDCl₃, 400 MHz) δ: 1.22 (6H, s), 2.75 (2H, s), 3.63 (2H, s), 7.12-7.30 (4H, m).

### MS (ESI): m/z 209 [M+H]⁺

### Preparation 4

### {3-[2-(2-Chloro-acetylamino)-2-methyl-propyl]-phenyl}-acetic acid

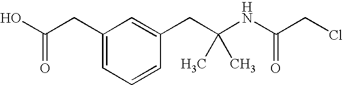

2-Chloroacetonitrile (1.63 kg, 21.62 moles) was added to a solution of the alcohol from preparation 3 (3.00 kg, 14.41 moles), in dichloromethane (15 L). The resulting solution was treated with acetic acid (2.6 kg, 43.23 moles) maintaining the temperature between 5° C. and 10° C. The resulting solution was treated with concentrated sulfuric acid (2.83 kg, 28.82 moles) maintaining the temperature between 5° C. and 10° C. The mixture was warmed to 20° C. and after 90 minutes the reaction mixture was added to cold water (30 L) maintaining the temperature below 10° C. The mixture was stirred for 30 minutes at 5 to 10° C. then at 20° C. for 30 minutes. The layers were separated and the aqueous layer was extracted with further dichloromethane (15 L). The combined dichloromethane layers were distilled down to 8 L volume at atmospheric pressure. The concentrate was treated with n-heptane (27 L) and toluene (3 L) and concentrated in vacuo to remove residual dichloromethane. The resulting slurry was granulated at 20° C. for 2 hours and the solid precipitate isolated by filtration and washed with n-heptane (2×3 L) to give the title compound as an off-white solid (3.76 kg).

¹H NMR (CDCl₃, 400 MHz) δ: 1.36 (s, 6H), 3.02 (s, 2H), 3.62 (s, 2H), 3.95 (s, 2H), 6.19 (m, 1H), 7.06-7.31 (m, 4H) ppm.

MS (electrospray): m/z 282 [M−H]⁻

### Preparation 5

### [3-(2-amino-2-methyl-propyl)-phenyl]-acetic acid ethyl ester

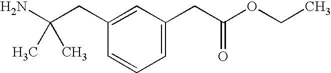

A mixture of the amide from preparation 4 (151.4 g, 534 mmol), thiourea (48.7 g, 640 mmol) and acetic acid (303 ml) in ethanol (1.5 L) was heated to reflux under a nitrogen atmosphere for 5 hours. The reaction mixture was allowed to cool to room temperature and the suspension concentrated in vacuo. The residues were azeotroped with toluene (2×900 mL) then treated with ethanol (1.5 L) and stirred for 1 hour. The solid precipitate was removed by filtration, and the filtrate cooled in an ice bath, treated with 98% sulfuric acid (227 mL) and stirred for 1 hour at ambient temperature. The solution was concentrated in vacuo to remove most of the ethanol and adjusted to pH9 using aqueous sodium hydrogen carbonate. The solid precipitate was removed by filtration and washed with water (300 mL) then ethyl acetate (1.0 L). The layers of the combined biphasic filtrate and washes were separated and the aqueous layer re-extracted with ethyl acetate (1.0 L+500 mL). The combined ethyl acetate extracts were dried over magnesium sulfate, filtered and concentrated in vacuo to give the title compound as a brown oil (89.5 g).

¹H NMR (DMSO-d₆, 400 MHz) δ: 0.99 (s, 6H), 1.16 (t, 3H), 2.59 (s, 2H), 3.61 (s, 2H), 4.06 (q, 2H), 7.06 (m, 3H), 7.21 (m, 1H)

### Preparation 5a

### [3-(2-amino-2-methyl-propyl)-phenyl]-acetic acid ethyl ester, di-p-toluoyl-L-tartrate salt

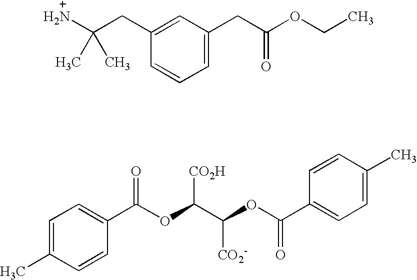

A solution of the amine from preparation 5 (assuming 9.45 mol) in acetonitrile (24.8 L) was treated with a solution of di-p-toluoyl-L-tartaric acid (3.65 kg, 9.45 moles) in acetonitrile (18.6 L). The resulting slurry was stirred for 15 hours at 20° C. and the solid precipitate was isolated by filtration and washed with acetonitrile (2×6.2 L) to give the title compound as a white solid (5.72 kg).

¹H NMR (DMSO-d₆, 400 MHz) δ: 1.13 (s, 6H), 1.17 (t, 3H), 2.34 (s, 6H), 2.78 (s, 2H), 3.63 (s, 2H), 4.06 (q, 2H), 5.61 (s, 2H), 7.02 (d, 2H), 7.15 (d, 1H), 7.25 (m, 5H), 7.80 (d, 4H)

### Preparation 5

### [3-(2-Amino-2-methyl-propyl)-phenyl]-acetic acid ethyl ester

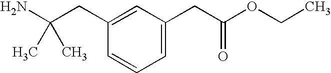

A solution of potassium carbonate (6.232 Kg, 45.1 mol) in water (35.04 L) was added to a suspension of the salt from preparation 5a (7.008 Kg, 11.272 mol) in propionitrile (35.04 L) and stirred until the entire solid had dissolved. The phases were then separated and the propionitrile phase washed with water (17.52 L). The solution was reduced in volume under reduced pressure to approximately 3.70 Kg to give the title compound as a propionitrile solution. A sample (20 mL) was removed and concentrated to dryness to obtain a weight/weight assay; the yield was shown to be 92%.

¹H NMR (DMSO-d₆, 400 MHz) δ: 0.99 (s, 6H), 1.16 (t, 3H), 2.59 (s, 2H), 3.61 (s, 2H), 4.06 (q, 2H), 7.06 (m, 3H), 7.21 (m, 1H)

### Alternative Procedure for the Preparation of the Product of Preparation 5

A solution of the amide from preparation 14 (3.10 kg, 9.945 moles) in ethanol (34.1 L) was treated with thiourea (0.91 kg, 11.93 moles) and acetic acid (6.2 L) and heated at reflux for 4 hours. The mixture was cooled and the solid precipitate was removed by filtration and washed with ethanol (3.1 L). The combined filtrate and wash were concentrated down to 8 L volume in vacuo, and azeotroped down to 8 L in vacuo with toluene (31.0 L and 24.8 L). The resulting mixture was treated with water (9.3 L) and 2M aqueous sodium carbonate solution (7.5 L) and extracted with dichloromethane (31.0 L and 15.5 L). The combined dichloromethane extracts were concentrated down to 8 L volume at atmospheric pressure, treated with acetonitrile (12.4 L) and concentrated down to 8 L volume in vacuo. The concentrate was diluted with acetonitrile (24.8 L) and used directly in preparation 5a.

### Preparation 6

### N-{2-(benzyloxy)-5-[(1R)-2-bromo-1-hydroxyethyl]phenyl}methane sulfonamide

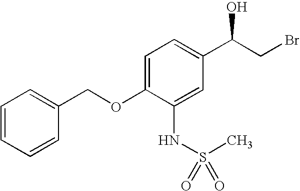

Pyridine (18.4 ml; 227.2 mmol) was added to a solution of (1R)-1-[3-amino-4-(benzyloxy)phenyl]-2-bromoethanol (Org. Process Research and Development, 1998, 2, 96) (36.59 g; 113.6) in THF (160 ml). Methanesulfonyl chloride (10.5 mL; 136.3 mmol) was then added and the reaction mixture was stirred at 20 to 25° C. for 2 hours. The reaction was quenched with 1M hydrochloric acid (180 mL) and then extracted with toluene (180 mL). The toluene solution was then washed with water (2×90 mL). The toluene solution was then concentrated under reduced pressure at 45° C. to 110 mL and the solution was then cooled to room temperature (20-25° C.) and stirred for one hour, the mixture was then cooled to 10-15° C. and stirred for 1 hour. The precipitate was collected by filtration, washed with toluene (2×10 mL) to give the title compound as a pink solid (37.95 g).

¹H NMR (DMSO-d₆, 400 MHz) δ: 2.93 (s, 3H), 3.52-3.66 (m, 2H), 4.74 (m, 1H), 5.19 (s, 2H), 7.11 (d, 1H), 7.19-7.22 (m, 1H), 7.33-7.36 (m, 2H), 7.40-7.43 (m, 2H), 7.56 (d, 2H), 8.95 (s, 1H) ppm.

MS (electrospray): m/z 398/400 [M−H]−

### Alternative Procedures for the Preparation of the Product of Preparation 6

The product of preparation 6 can be prepared by stereoselective enzymatic reduction of N-[2-benzyloxy-5-(2-bromo-acetyl)-phenyl]-methanesulfonamide (Journal of Medicinal Chemistry, 1967, 10, 462 and Journal of Medicinal Chemistry, 1980, 23, 738), as described in the Journal of the American Oil Chemists' Society 1998, 75, 1473 as well as in the examples below.

A biotransformation can be achieved by those skilled in the art by contacting the substance to be transformed, and other necessary reactants, with the enzymes derived from a variety of living organisms under conditions suitable for a chemical interaction to occur. Subsequently, the products of the reaction are separated and those of interest are purified for elucidation of their chemical structure and physical and biological properties. The enzymes can be present as purified reagents, be in crude extracts or lysates, or be in intact cells and can be in solution, be in suspension (e.g., intact cells), be covalently attached to a supporting surface, or be embedded in a permeable matrix (e.g., agarose or alginate beads). The substrate and other necessary reactants (e.g., water, air, cofactors) are supplied as the chemistry dictates. Generally, the reaction is carried out in the presence of one or more liquid phases, aqueous and/or organic, to promote mass transfer of the reactants and products. The reaction can be conducted aseptically or not. The conditions for monitoring the progress of the reaction and the isolation of the products of the reaction will vary according to the physical properties of the reaction system and the chemistry of the reactants and products.

For whole-cell biocatalysis, a nutrient medium (e.g., IOWA Medium: dextrose, yeast extract, dipotassium hydrogen phosphate, sodium chloride, soybean flour, water; adjusted to neutral pH) is added to one or more culture vessels (e.g., fermentation tubes or flasks) that are then steam-sterilized. Each vessel is aseptically inoculated with growth from an agar culture, a suspension of washed cells or spores, or broth from a liquid nutrient medium culture of the biotransforming microorganism. The vessels are mounted on a shaker designed for fermentation and shaken (e.g., rotary operation at 100-300 rpm) at an appropriate temperature (e.g., 20-40° C.) long enough to promote the growth of the microorganism to a suitable population size (e.g., 1-3 days). The compound to be transformed (i.e., substrate) is dissolved in water or a suitable water-miscible solvent (e.g., dimethylsulfoxide, dimethylformamide, ethyl alcohol, methyl alcohol). To each of the biotransformation vessels, the resulting solution is aseptically added to achieve the desired concentration of substrate. The dosed vessels are mounted on the shaker and shaken as before, until the substrate has been converted to product[s] by microbial metabolism (e.g., 1-10 days).

Isolated enzymes can be mixed with suitable agitation in a suitable buffer (e.g. potassium phosphate) with any required co-factors and the substrate, with or without organic solvent, at a suitable temperature (25-37° C.) and duration for biocatalysis. Many enzymes can be screened at once in microtiter plates. Enzymes are dissolved in a suitable buffer and distributed into individual wells of a microtiter plate. Enzymes can be frozen (−80° C.) or used immediately. To screen, additional buffer is added to each well along with the substrate and any co-factors required for the enzyme function (e.g., NADPH). The plate is then mixed (e.g., Eppendorf thermomixer) as mentioned above.

The contents of the biotransformation vessel can mechanically treated (e.g, by filtration or centrifugation) to separate solids from the aqueous phase and/or extracted at a pH optimal for extraction of the desired compounds (water-immiscible organic solvents include, but are not limited to, methylene chloride or ethyl acetate). Samples can be analyzed by HPLC or other suitable technique.

The following are two examples of laboratory-scale screening methods for carrying out biotransformations that can be exercised by those skilled in the art to produce the compound of interest.

### Alternative Process 1 for the Synthesis of Preparation 6

### Stereoselective Microbial Reduction of N-[2-benzyloxy-5-(2-bromo-acetyl)-phenyl]-methanesulfonamide to the Corresponding (R)-Alcohol

The incubations were carried out in 2.5 ml of IOWA Medium (anhydrous dextrose, 20 g; yeast extract, 5 g; dipotassium hydrogen phosphate, 5 g; sodium chloride, 5 g; soybean flour, 5 g; distilled water, 1 L; adjusted to pH 7.0 with 1N hydrochloric acid, steam-sterilized for 15 minutes at 15 psig and 121° C.) in 16×125 mm glass tubes with stainless steel Morton closures. Tubes were aseptically inoculated with 0.025 mL of a cryogenically stored (−80° C.) stock of Candida magnoliae ATCC 56463 mycelium. The inoculated tubes were mounted at a slight angle on a rotary shaker (2-inch throw) and shaken at 210 rpm and 29° C. for 2 days. N-[2-Benzyloxy-5-(2-bromo-acetyl)-phenyl]-methanesulfonamide (i.e., substrate) was dissolved in dimethylsulfoxide (10 mg/mL). Substrate was added to each tube to give an initial substrate concentration of 0.1 mg/mL up to 1 mg/mL. The dosed tubes were shaken at 210 rpm and 29° C. for an additional 6 days. At the end of the 6-day biotransformation period, the contents of the biotransformation tubes were extracted with 4 mL ethyl acetate. The organic phases were concentrated under nitrogen. The residues were reconstituted in an appropriate quantity of methanol for chiral HPLC analysis. At 1 mg/mL substrate concentration, the reaction yield to the (R)-alcohol was 33%, >99% ee.

Chiral HPLC analytical method:

Instrument: Waters 2695 HPLC system with 996 photodiode array detector.

Column: Chiralpak AD-H, 4.6×150 mm.

Mobile phase: methanol:ethanol [1:1] at 1 ml/min.

Detection: PDA maxplot: 210-400 nm.

The (R)-alcohol eluted at 2.95 minutes; the substrate eluted at 6.02 minutes.

### Alternative Process 2 for the Synthesis of Preparation 6

### Stereoselective Enzymatic Reduction of N-[2-benzyloxy-5-(2-bromo-acetyl)-phenyl]-methanesulfonamide to the Corresponding (R)-Alcohol

50 mg of KRED-130 from BioCatalytics (Pasadena, Calif.) was dissolved in 1.5 mL of buffer (50 mM potassium phosphate buffer, 0.1M potassium chloride, 0.5 mM dithiothreitol, pH 6.0) and 0.030 mL distributed into a well as part of a ketoreductase screening plate. The plates had been frozen at −80° C. with a polypropylene cover and one thawed prior to use for this experiment. 0.42 mL of buffer (above) was added to each screening well along with 0.1 mg of the substrate (0.01 mL of a 10 mg/mL DMSO stock solution) and NADPH (0.040 mL of a 100 mg/mL water stock solution). The plate was incubated on an Eppendorf thermomixer R at 30° C. and 750 rpm for 24 hours. Each well was extracted with 0.8 mL ethyl acetate then centrifuged (Damon IEC centrifuge (CRU5000), 2200 rpm, 3 minutes). 0.7 mL was removed from each well into a new microtiter plate. The organic phase was dried under nitrogen then reconstituted in methanol for HPLC analysis (above). The desired (R)-alcohol was produced at 57% yield, >99% ee.

### Preparation 7

### N-[2-(benzyloxy)-5-((1R)-2-bromo-1-{[tert-butyl(dimethyl)silyl]oxy}ethyl)phenyl]methanesulfonamide

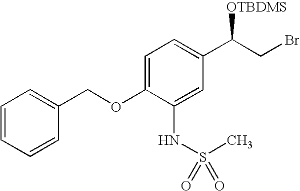

A solution of the bromide of preparation 6 (10 g; 25.0 mmol) was dissolved in dichloromethane (20 mL) and then imidazole (4.58 g; 37.5 mmol) was added followed by tert-butyldimethylsilyl chloride (5.27 g; 35.0 mmol). The reaction mixture was heated to reflux for 1 hour and then cooled to 30° C. The mixture was diluted with isopropyl acetate (80 mL) and then quenched with 2M hydrochloric acid (50 mL) and stirred vigorously for 10 minutes. The phases were separated and the organic phase was washed with water (50 mL). The organic phase was then reduced in volume under reduced pressure at 45° C. to 25 to 30 mL. The solution was then cooled to room temperature and a suspension quickly formed and was stirred at room temperature for 30 minutes. Heptane (20 mL) was then added over 10 minutes and the suspension was cooled to 5 to 10° C. and stirred for 1 hour. The suspension was then filtered and washed on the filter paper with heptane (2×10 mL) to give the title compound as a white solid (11.05 g).

¹H NMR (CDCl₃, 400 MHz) δ: −0.07 (s, 3H), 0.11 (s, 3H), 0.89 (s, 9H), 2.91 (s, 3H0, 4.80-4.83 (m, 1H), 6.80 (bs, 1H), 6.98 (d, 1H), 7.12 (d, 1H), 7.36-7.44 (m, 5H), 7.52-7.54 (m, 1H) ppm.

### Preparation 8

### N-[2-(benzyloxy)-5-((1R)-2-bromo-1-{[tert-butyl(dimethyl)silyl]oxy}ethyl)phenyl]-dimethanesulfonamide

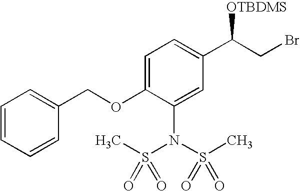

N-[2-(Benzyloxy)-5-((1R)-2-bromo-1-{[tert-butyl(dimethyl)silyl]oxy}ethyl)phenyl]methane sulfonamide prepared as described in preparation 7 (20.0 g; 39.2 mmol) and diisopropylethylamine (24 mL; 138 mmol) were combined in acetonitrile (100 mL) and cooled to about 5° C. Methanesulfonyl chloride (9.0 mL; 118.8 mmol) was added over about 10 minutes and the resultant mixture was stirred for about 1 hour at 5° C. Water (300 mL) was added and the resultant slurry granulated for 15 minutes then filtered and dried at 40° C. under vacuum to provide the title compound (23.3 g) as a pale yellow solid.

¹H NMR (CDCl₃, 400 MHz) δ: −0.06 (s, 3H), 0.12 (s, 3H), 0.90 (s, 9H), 3.31 (s, 6H), 3.40-3.50 (m, 2H), 4.83 (dd, 1H), 5.14 (s, 2H), 7.05 (d, 1H), 7.32-7.42 (m, 5H), 7.46-7.50 (m, 2H) ppm.

### Preparation 9

### N-[2-(benzyloxy)-5-((1R)-2-bromo-1-hydroxyethyl)phenyl]-dimethanesulfonamide

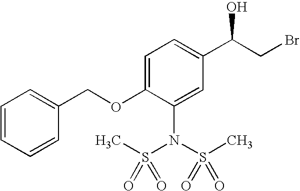

The silyl ether from preparation 8 (19.2 g; 32.4 mmol) was suspended in a mixture of tetrahydrofuran (40 mL) and methanol (2 mL). Triethylamine trihydrofluoride (9 mL; 55.2 mmol) was added and the resultant solution was stirred for 30 hours at ambient temperature. The reaction was quenched with aqueous ammonia (35%, 20 mL) and the product was extracted into ethyl acetate (2×30 mL). The combined organic phases were washed with saturated aqueous sodium hydrogen carbonate and water, dried with anhydrous MgSO₄, filtered and concentrated to dryness. The residue was then slurried in ethyl acetate (40 mL) for 2 hours, after which time the product was isolated by filtration, washing with ethyl acetate (10 mL) and tert-butyl methyl ether (20 mL) to give the title compound (11.3 g) as a white solid.

¹H NMR (CDCl₃, 400 MHz) δ: 3.33 (s, 6H), 3.51 (dd, 1H), 3.63 (dd, 1H), 4.90 (dd, 1H), 5.16 (s, 2H), 7.08 (d, 1H), 7.33-7.45 (m, 5H), 7.46-7.50 (m, 2H) ppm.

### Preparation 10

### N—[(R)-2-benzyloxy-5-oxiranyl-phenyl]-dimethanesulfonamide

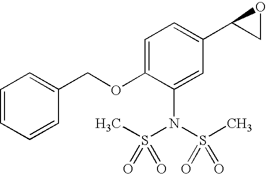

To a solution of the bromohydrin from preparation 9 (6.0 g; 12.5 mmol) in a mixture of methanol (30 mL) and tetrahydrofuran (30 mL) was added potassium carbonate (2.25 g; 16.3 mmol) and the resultant mixture was stirred at ambient temperature for about 18 hours. The reaction was quenched into water (60 mL) and extracted with propionitrile (2×60 mL). The combined propionitrile layers were washed with water (100 mL), dried with anhydrous MgSO₄, filtered and concentrated to yield the title compound (4.98 g) as a pale yellow solid that was used without further purification.

¹H NMR (CDCl₃, 400 MHz) δ: 2.76 (dd, 1H), 3.13 (dd, 1H), 3.31 (s, 3H), 3.33 (s, 3H), 3.83 (m, 1H), 5.15 (s, 2H), 7.06 (d, 1H), 7.22 (d, 1H), 7.31-7.44 (m, 4H), 7.46-7.50 (m, 2H) ppm.

### Preparation 11

### tert-Butyl (3-bromobenzyl)carbamate

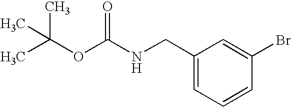

Triethylamine (6.57 L; 46.7 mol) was added to 3-bromobenzylamine hydrochloride (9.9 Kg; 44.5 mol) in ethyl acetate (39.6 L) and the resulting mixture was stirred for 30 minutes at 20 to 25° C. and was then cooled to 0° C. A solution of di-tert-butyl dicarbonate (10.7 Kg; 49 mol) in ethyl acetate (19.8 L) was then added over 30 minutes at such a rate as to maintain the temperature between 0° C. and 20° C. The reaction mixture was then stirred at 20 to 25° C. for 2 hours, water (29.7 L) was then added and the mixture was stirred vigorously for 10 minutes and then the phases were separated. The ethyl acetate phase was distilled and replaced with heptane under reduced pressure at 35 to 45° C. to a final volume of approximately 40 L and then the solution was cooled to 0° C. over 2 hours. The resulting suspension was stirred at 0° C. for 12 hours, then the product was collected by filtration, washing with heptane (2×3.37 L) to provide the title compound as a white solid (10.26 Kg).

¹H NMR (400 MHz, CDCl₃) δ: 1.46 (s, 9H), 4.25-4.32 (m, 2H), 4.75-4.90 (bs, 1H), 7.16-7.22 (m, 2H), 7.39 (dt, 1H), 7.43 (bs, 1H) ppm.

### Preparation 12

### tert-Butyl [(4′-hydroxybiphenyl-3-yl)methyl]carbamate

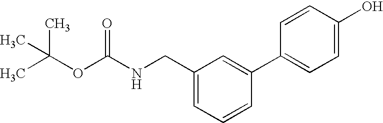

Nitrogen was bubbled through a stirred solution of the bromide from preparation 11 (5.12 Kg; 17.9 mol), 4-hydroxyphenylboronic acid (2.71 Kg; 19.7 mol) and sodium carbonate (2.848 Kg; 26.8 mol) in a mixture of 1,4 dioxane (25.6 L) and demineralised water (25.6 L) at 20 to 25° C. for 1 hour. Then 1,1′-bis(diphenylphosphino)ferrocenyl palladium(II)chloride (14.6 g; 0.0179 mol) was added to the mixture and the nitrogen bubbling was continued for a further 30 minutes. Subsequently, the reaction was heated at 65 to 70° C. under a nitrogen blanket for 2 hours. The reaction was cooled to 20 to 25° C., ethyl acetate (41 L) was added and the resulting mixture was stirred vigorously for 10 minutes, the phases were then separated. The organic phase was washed with a solution of citric acid (1.9 Kg) in demineralised water (18.9 L) followed by a solution of sodium chloride (3.15 Kg) in demineralised water (18.9 L). The ethyl acetate solution was treated with activated carbon (Darco KB 100 mesh, wet powder; 5.12 Kg) and stirred for 12 hours. The resulting slurry was then filtered through Arbocel and the cake was washed with methanol (25.6 L). The combined filtrate was distilled and replaced with toluene under reduced pressure at 40 to 50° C. to a final volume of approximately 15 L. The solution was then cooled to 10° C. over 2 hours and the resulting suspension was stirred at 10° C. for 12 hours. The product was isolated by filtration and washed with cyclohexane (2×2.56 L) to provide the title compound as a white solid (4.26 Kg).

¹H NMR (400 MHz, CDCl₃) δ: 1.47 (s, 9H), 4.33-4.41 (m, 2H), 4.87-4.94 (bs, 1H), 6.89 (d, 2H), 7.21 (d, 1H), 7.37 (dd, 1H), 7.43-7.45 (m, 4H) ppm.

MS (electrospray) m/z 298 [M−H]⁻, 322 [M+Na]⁺

### Preparation 13

### 3′-(Aminomethyl)biphenyl-4-ol hydrochloride

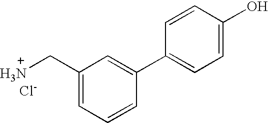

A solution of hydrogen chloride in 1,4-dioxan/water (4M, 64.7 L; 135 mol) was added to a solution of the phenol from preparation 12 (8.09 Kg; 27 mol) in 1,4-dioxan (16.15 L) over 20 minutes and the resulting mixture was stirred at 20 to 25° C. for 1 hour. The suspension was concentrated under reduced pressure at 40 to 45° C. to approximately 40 L and was stirred for 12 hours at 20 to 25° C. The precipitate was collected by filtration and washed with 1,4-dioxan (2×4.05 L). The resulting filter cake was added to acetonitrile (80.9 L) and heated at reflux for 2 hours. The precipitate was isolated by filtration and washed with acetonitrile (2×4.05 L) to provide the title compound as a white solid (3.65 Kg; 57%).

The aqueous 1,4-dioxane liquors were distilled and replaced with fresh 1,4-dioxane until the vapour temperature was greater than 100° C. and the reaction volume was ˜40 L. The reaction mixture was cooled down to 20 to 25° C., granulated for 18 hours and the crude product was isolated by filtration. The resulting filter cake was added to acetonitrile (40 L) and heated at reflux for 2 hours. The resulting precipitate was isolated by filtration and washed with acetonitrile (2×4.05 L) to provide a second crop of the title compound as a pale brown solid (2.36 Kg; 37%).

¹H NMR (400 MHz, CD₃OD) δ: 4.17 (s, 2H), 6.87 (d, 2H), 7.34 (d, 1H), 7.45-7.50 (m, 3H), 7.61 (d, 1H), 7.65 (s, 1H) ppm.

MS (electrospray) m/z 198 [M−H]⁻, 200 [M+H]⁺

### Preparation 14

### {3-[2-(2-Chloro-acetylamino)-2-methyl-propyl]-phenyl}-acetic acid ethyl ester

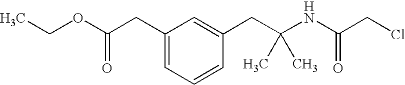

A solution of the acid from preparation 4 (3.76 kg, 13.24 moles) in ethanol (30.1 L) was treated with concentrated sulfuric acid (130 g, 1.31 moles) and heated at reflux for 90 minutes. The cooled solution was adjusted to ˜pH5 using 1.0M aqueous sodium hydrogen carbonate solution (2.0 kg). The mixture was concentrated down to 8 L volume in vacuo, diluted with toluene (11.7 L) and concentrated down to 12 L volume in vacuo. The concentrate was diluted with toluene (25.8 L), washed with water (22.6 L) and the aqueous layer was re-extracted with further toluene (15.0 L). The combined toluene layers were concentrated down to 8 L in vacuo. The concentrate was held at 35° C. and treated with n-heptane (15.0 L) maintaining the temperature above 30° C. The mixture was cooled and the resulting slurry was granulated at 20° C. for 2 hours. The solid precipitate was isolated by filtration and washed with n-heptane (2×3.76 L) to give the title compound as a white solid (3.15 kg).

¹H NMR (DMSO-d₆, 400 MHz) δ: 1.14 (t, 3H), 1.19 (s, 6H), 2.95 (s, 2H), 3.59 (s, 2H), 3.94 (s, 2H), 4.07 (q, 2H), 7.00 (m, 2H), 7.09 (d, 1H), 7.20 (t, 1H), 7.59 (s, 1H)

### Preparation 15

### Ethyl [3-(2-tert-butoxycarbonylamino-2-methylpropyl)phenyl]acetate

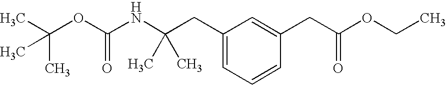

The amine from preparation 5 (48.0 g; 204 mmol) was added to a solution of di-tert-butyl dicarbonate (55.0 g; 252 mmol) and 4-dimethylaminopyridine (1.5 g; 12.3 mmol) in THF (50 mL) over approximately 30 min and the resulting solution was stirred at ambient temperature under nitrogen for 23 h. The reaction mixture was then partitioned between ethyl acetate (100 mL) and hydrochloric acid (1.5M, 150 mL) and the phases were separated. The organic phase was washed with water (100 mL) and brine (50 mL), dried over anhydrous MgSO₄ and concentrated to give the title compound (65.8 g) as a dark brown oil, which was used without further purification.

¹H NMR (CDCl₃, 400 MHz) δ: 1.22-1.24 (m, 9H), 1.47 (s, 9H), 2.96 (s, 2H), 3.57 (s, 2H), 4.13 (q, 2H), 4.27 (s, 1H), 7.05 (m, 2H), 7.15 (m, 1H), 7.22 (m, 1H) ppm

### Preparation 16

### [3-(2-tert-Butoxycarbonylamino-2-methylpropyl)phenyl]acetic acid

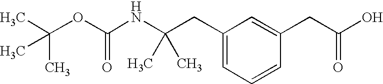

Sodium hydroxide (16.0 g; 400 mmol) and water (100 mL) were added to a cooled solution of the ethyl ester from preparation 15 (64.7 g; 193 mmol) in THF (100 mL) and the resulting solution was stirred at ambient temperature for about 16 h. The solution was then acidified to pH 1 with hydrochloric acid and the product was extracted into ethyl acetate (2×200 mL). The combined organic extracts were washed with water and brine, dried over anhydrous MgSO₄ and concentrated to give the title compound (57.3 g) as a thick brown oil. Recrystallisation from toluene/heptane gave the product as an off-white solid.

¹H NMR (CDCl₃, 400 MHz) δ: 1.25 (s, 6H), 1.47 (s, 9H), 2.96 (s, 2H), 3.61 (s, 2H), 7.07 (m, 2H), 7.15 (m, 1H), 7.23 (m, 1H) ppm.

### Alternative Process for the Preparation of the Product of Preparation 16

Diisopropylethylamine (210 mL; 1.21 mol) was added to a suspension of the salt from preparation 5a (250 g; 0.40 mol) in propionitrile (1.0 L), giving a pale yellow solution. A solution of di-tert-butyl dicarbonate (97 g; 0.44 mol) in propionitrile (250 mL) was added and the resulting pale yellow solution was stirred at ambient temperature for 21 h. Water (250 mL) was added and the mixture was stirred for 30 min. The phases were separated and the organic phase was washed successively with 10% aqueous citric acid (500 mL), water (300 mL), saturated aqueous sodium hydrogen carbonate (500 mL) and brine (500 mL). The organic phase was then concentrated to a dark orange oil and dissolved in a mixture of tetrahydrofuran (250 mL) and water (250 mL). Sodium hydroxide (80 g; 2.0 mol) was added and the resulting mixture was stirred at ambient temperature for 91 h. Toluene (400 mL) was added and the mixture was stirred for 30 min and then the phases were separated. The organic phase was extracted with a mixture of water (200 mL) and saturated aqueous sodium hydrogen carbonate (100 mL). The combined aqueous phase was then adjusted to pH 1 with concentrated hydrochloric acid and extracted with ethyl acetate (2×250 mL). The combined ethyl acetate extracts were washed with water (2×200 mL) and then concentrated to dryness. The resulting oil was dissolved in refluxing toluene (100 mL) and heptane (˜400 mL) was added. The mixture was cooled to ambient temperature and granulated for 3 h. The solid was isolated by filtration, washing with heptane (2×200 mL) and dried in a vacuum oven at 40° C. to give the title compound (110.9 g; 90%) as a pale yellow solid.

### Preparation 17

### tert-butyl-[2-(3{[(4′-hydroxybiphenyl-3-ylmethyl)-carbamoyl]-methyl}-phenyl)-1,1-(dimethyl)ethyl]carbamate

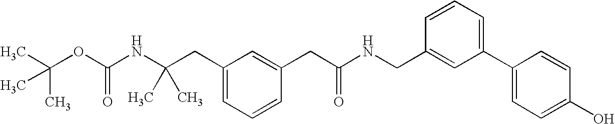

A mixture of the acid from preparation 16 (25 g; 81.3 mmol), the amine hydrochloride from preparation 13 (18.2 g; 77.3 mmol), 4-dimethylaminopyridine (100 mg; 0.81 mmol) and diisopropylethylamine (22.1 g; 170.8 mmol) in acetonitrile (125 mL) was stirred at ambient temperature under nitrogen while 1-(3-dimethylaminopropyl)-3-ethylcarbodiimide hydrochloride (17.15 g; 89.5 mmol) was then added and the mixture was stirred for 18 hours at ambient temperature. Water (190 mL) was added and the resulting suspension was stirred for 1.5 h. The solid was then isolated by filtration, washing with water (100 mL) and dried under suction for 20 min. The damp filter cake was slurried in 10% aqueous citric acid (100 mL) for 1 h, The solid was isolated by filtration, washing with water (100 mL) to give the title compound (31.0 g; 82%) as a white solid.

¹H NMR (DMSO-d₆, 400 MHz) δ: 1.11 (s, 6H), 1.39 (s, 9H), 2.85 (s, 2H), 3.43 (s, 2H), 4.30 (d, 2H), 6.25 (s, 1H), 6.82 (d, 2H), 6.98 (d, 1H), 7.03 (s, 1H), 7.09-7.20 (m, 3H), 7.30 (t, 1H), 7.35-7.42 (m, 4H), 8.50 (s, 1H), 9.50 (s, 1H).

### Preparation 18

### {3-[2-(2,2,2-Trichloro-acetylamino)-2-methyl-propyl]-phenyl}-acetic acid

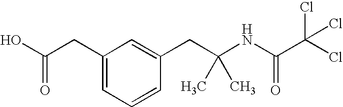

Trichloroacetonitrile (20 g, 0.14 mol) was added to a solution of the alcohol from preparation 3 (20 g, 0.09 mol), in acetic acid (40 mL). The resulting solution was cooled to 0° C., treated with concentrated sulfuric acid (98%; 30 mL), and the reaction mixture allowed to warm gradually to room temperature. After 4 hours the reaction mixture was poured onto ice/water (400 mL) and the solution was extracted with isopropyl acetate (2×200 mL). The combined organic layers were washed with demineralised water (120 mL), and then concentrated in vacuo affording a viscous brown oil. The oil was then treated with toluene (100 mL) and concentrated. The residues were then treated with heptane (100 mL) and filtered under vacuum to give the title product as an off white solid (28.32 g).

¹H NMR (CD₃OD, 400 MHz) δ: 1.39 (s, 6H), 3.07 (s, 2H), 3.56 (s, 2H), 7.07-7.45 (m, 1H), ppm.

### Preparation 19

### 2,2,2-Trichloro-N-[2-(3-{[4′-hydroxy-biphenyl-4-ylmethyl)-carbamoyl]-methyl}-phenyl)-1,1-dimethyl-ethyl]acetamide

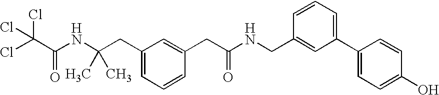

The product of preparation 13 (19.8 g, 0.085 mol), 1-(3-dimethylaminopropyl)-3-ethylcarbodiimide hydrochloride (24.45 g, 0.13 mol), 1-hydroxybenzotriazole (17.2 g, 0.13 mol), triethylamine (42.9 g, 0.42 mol) and the product from preparation 18 (30 g, 0.085 mol) were suspended in ethyl acetate and heated at 40° C. for 20 hours. The ethyl acetate solution was washed with water (4×150 mL) and then concentrated to give the title compound as a brown solid (33.7 g).

¹H NMR (CD₃OD, 400 MHz) δ: 1.34 (s, 6H), 3.01 (s, 2H), 3.53 (s, 2H), 4.40 (s, 2H), 6.82-7.41 (m, 12H) ppm.

### Preparation 20

### 2-[3-(2-Amino-2-methylpropyl)-phenyl]-N-[(4′-hydroxybiphenyl-3-yl)methyl]acetamide

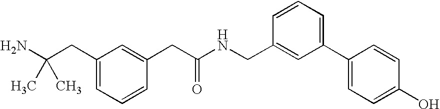

A suspension of the Boc-protected amine from preparation 17 (28.0 g; 57.3 mmol) in ethanol (100 mL) was treated with hydrochloric acid (4M in dioxane, 35 mL; 80 mmol) and the reaction was stirred for about 100 hours at ambient temperature. The reaction mixture was poured into a mixture of aqueous ammonia (35%, 30 mL) and water (200 mL). The product was then and extracted with propionitrile (2×50 mL) and n-butanol (100 mL). The combined organic phases were washed with water, dried with anhydrous magnesium sulfate, filtered and concentrated. The residue was slurried in acetone (100 mL) for about 18 hours and the resultant suspension was filtered and dried to yield the title compound (13.4 g) as an off-white solid.

¹H NMR (CD₃OD, 400 MHz) δ: 1.09 (s, 6H), 2.66 (s, 2H), 3.56 (s, 2H), 4.41 (s, 2H), 6.82 (d, 2H), 7.08-7.15 (m, 3H), 7.20-7.42 (m, 8H).

MS (electrospray) m/z 389 [M+H]⁺, 372 [M−H₂O]⁺

### Alternative Processes for the Preparation of the Product of Preparation 20

Alternative Process 1:

A suspension of the protected amine from preparation 17 (31.0 g; 63.4 mmol) in dichloromethane (150 mL) was stirred under an inert atmosphere while trifluoroacetic acid (50 mL; 649 mmol) was added. The resulting pale orange-brown solution was stirred for 1.5 h, and then concentrated under reduced pressure to give a thick brown oil. The oil was treated with a mixture of water and concentrated aqueous ammonia (9:1, ˜250 mL) until pH 12 was reached, then the mixture was extracted with a mixture of ethyl acetate and methanol (9:1, 2×150 mL). The combined organic extracts were washed with water and then concentrated under reduced pressure. The resultant foam was refluxed in acetone (500 mL) for 1 h and then cooled to ambient temperature and granulated overnight. The solid was isolated by filtration, washing with acetone, and dried at 40° C. in a vacuum oven to give the title compound (13.42 g; 54%) as a white solid.

Alternative Process 2:

The product from preparation 19 (33 g, 0.06 mol) was dissolved in a mixture of 4M aqueous potassium hydroxide (78.6 mL) and ethanol (78.6 mL) and was stirred at 50° C. for 24 hours. The mixture was partially concentrated under vacuum (to about 80 mL) and was then extracted with ethyl acetate (4×40 mL). The organic extracts were combined and concentrated in vacuo to give crude title product as a yellow oil (24.11 g). This material was suspended in acetone (120 mL), heated to reflux and the solution was cooled to room temperature over 10 hours and was granulated at 5° C. for 1 hour before filtering under vacuum, washing with acetone (25 mL) to give the title compound as a white solid (7 g).

Alternative Process 3:

1-Hydroxybenzotriazole hydrate (11.93 g; 0.08 mol), the amine hydrochloride from preparation 13 (45.78 g; 0.19 mol) and triethylamine (35.73 g; 0.35 mol) were sequentially added to a solution of the alcohol from preparation 3 (36.77 g; 0.18 mol) in dichloromethane (368 mL). The solution was stirred for 1 h and then 1-ethyl-3-(3-dimethylaminopropyl)carbodiimide hydrochloride (33.84 g; 0.18 mol) was added and the mixture was stirred for 2 hours at ambient temperature. Tetrahydrofuran (184 mL) was added and the resulting solution was washed sequentially with water (2×184 mL), 1M aqueous hydrochloric acid (2×184 mL), and 1M aqueous potassium hydrogen carbonate (2×184 mL). The organic solution was distilled and replaced with chloroacetonitrile (132 mL). Trifluoroacetic acid (331 mL) was added to the chloroacetonitrile solution and the resulting mixture was heated to 50° C. for 2 h. Dichloromethane (331 mL) was added and the organic phase was washed with water (2×662 mL) followed by 1M aqueous potassium hydrogen carbonate (2×331 mL). The organic solution was then distilled and replaced with acetic acid (404 mL). Thiourea (44 g; 0.58 mol) was added to an aliquot of this solution (250 mL) and the resulting suspension was heated to 100° C. for 3 h. The suspension was filtered, and the cake was washed with acetic acid (54 mL). The acetic acid solution was diluted with water (774 mL) and the aqueous layer was washed with a mixture of dichloromethane and methanol (9:1, 2×242 mL). Methanol (53 mL) was added to the aqueous phase and the pH was adjusted to >9 using concentrated aqueous ammonia (˜230 mL), keeping the temperature below 15° C. Dichloromethane (480 mL) was added and the mixture stirred for 30 min. The phases were then separated and the organic phase was distilled and replaced with acetone (˜440 mL). The resulting suspension was cooled to ambient temperature and stirred for 18 h, then granulated at 5° C. for 2 h. The product was collected by filtration, washing with acetone (2×45 mL) to provide the title compound as a pale yellow solid (11.39 g).

### Preparation 21

### 2-(3-{2-[((2R)-2-{4-Benzyloxy-3-[(dimethylsulfonyl)amino]phenyl}-2-hydroxyethyl)amino]-2-methylpropyl}phenyl)-N-[(4′-hydroxybiphenyl-3-yl)methyl]-acetamide

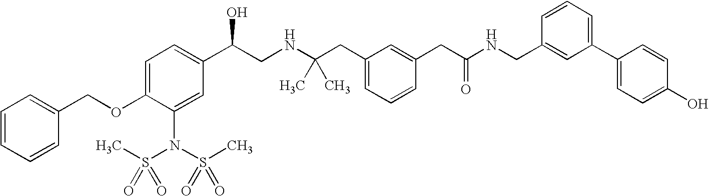

A mixture of the amine from preparation 20 (500 mg; 1.29 mmol) and the epoxide from preparation 10 (670 mg; 1.69 mmol) in butyronitrile (2 mL) was heated at reflux for 20 hours under an inert atmosphere. The mixture was then cooled to ambient temperature, and chromatographed directly on silica gel (40 g), eluting with methanol-dichloromethane (1:19 to 1:9) to provide the title compound (543 mg) as a waxy oil.

¹H NMR (CD₃OD, 400 MHz) δ: 1.00 (s, 3H), 1.03 (s, 3H), 2.66 (dd, 2H), 2.82 (m, 2H), 3.31 (s, 6H), 3.55 (s, 2H), 4.40 (s, 2H), 4.69 (dd, 1H), 5.16 (s, 2H), 6.82 (d, 2H), 7.03-7.54 (m, 18H).

MS (electrospray) m/z 786 [M+H]⁺

### Preparation 22

### 2-(3-{2-[((2R)-2-{4-Benzyloxy-3-[(methylsulfonyl)amino]phenyl}-2-hydroxyethyl)amino]-2-methylpropyl}phenyl)-N-[(4′-hydroxybiphenyl-3-yl)methyl]-acetamide

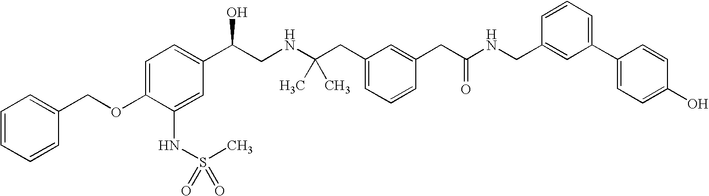

A solution of sodium hydroxide (500 mg; 12.5 mmol) in water (5 mL) was added to a solution of the bis-sulfonamide from preparation 21 (500 mg; 0.64 mmol) in ethanol (5 mL) and the resulting yellow solution was stirred for 14 days at ambient temperature. The mixture was then diluted with water (10 mL) and washed with dichloromethane (10 mL). The aqueous phase was adjusted to pH 1 with hydrochloric acid and extracted with propionitrile (2×20 mL). The combined propionitrile extracts were washed with water, dried with anhydrous MgSO₄, filtered and concentrated to give the title compound (272 mg) as a pale yellow glassy solid.

¹H NMR (CD₃OD, 400 MHz) δ: 1.03 (s, 3H), 1.05 (s, 3H), 2.68 (dd, 2H), 2.78-2.90 (m, 4H), 3.34 (s, 3H), 3.54 (s, 2H), 4.40 (s, 2H), 4.66 (dd, 1H), 5.18 (s, 2H), 6.81 (m, 2H), 7.01-7.40 (m, 16H), 7.43-7.48 (m, 2H).

MS (electrospray) m/z 708 [M+H]⁺

### Alternative Processes for the Preparation of the Product of Preparation 22

Alternative Process 1:

The crude silyl ether from preparation 24 (1.24 g; assume 1.7 mmol) was dissolved in a mixture of THF (5 mL) and methanol (1 mL). Triethylamine trihydrofluoride (0.5 mL; 3.1 mmol) was added and the mixture was stirred at ambient temperature for 8 h. The reaction was quenched with aqueous ammonia (35%, 10 mL) and extracted with propionitrile (2×20 mL). The combined propionitrile extracts were washed with water, dried with anhydrous MgSO₄, filtered and concentrated to give a brown foam. This was chromatographed on silica gel, eluting with methanol-dichloromethane (1:9) to yield the title compound (474 mg) as an off-white foam.

Alternative Process 2:

The crude silyl ether from preparation 25 (3.0 g; assume 3.6 mmol) was dissolved in THF (15 mL). Triethylamine trihydrofluoride (1.5 mL; 9.2 mmol) was added, followed by ethanol (0.5 mL) after 10 min. The pale orange solution was stirred at ambient temperature for 3 h, and then aqueous ammonia (35%, 10 mL) was added and the product was extracted into propionitrile (2×20 mL). The combined organic phases were washed with water, dried with anhydrous MgSO₄, filtered and concentrated to yield the title compound (2.6 g) as a pale brown foam (˜70% pure).

Alternative Process 3:

A mixture of the protected bromohydrin from preparation 7 (13.21 g; 25.7 mmol), the amine from preparation 20 (9.50 g; 24.4 mmol) and sodium hydrogen carbonate (4.11 g; 48.9 mmol) in n-butyl acetate (29 mL) was refluxed under nitrogen for 24 h. The mixture was cooled to ambient temperature and diluted with water (30 mL) and ethyl acetate (114 mL). The phases were separated and the organic phase was washed successively with 1M aqueous (L)-tartaric acid (25 mL), water-concentrated aqueous ammonia (3:1, 40 mL) and water (19 mL). Methanol (19 mL) was added, followed by triethylamine trihydrofluoride (4.5 mL; 27.6 mmol) and the resulting mixture was stirred at ambient temperature under nitrogen. After 1 h, a further aliquot of methanol (9.5 mL) was added. After 6 h, the reaction was quenched with a mixture of water and concentrated aqueous ammonia (3:1, 40 mL) and was stirred for 15 min. The phases were separated and the organic phase was washed with water (47.5 mL) and the ethyl acetate was distilled off under reduced pressure to give an n-butyl acetate solution of the title compound, which was used directly in preparation 22a.

### Preparation 22a

### 2-(3-{2-[((2R)-2-{4-Benzyloxy-3-[(methylsulfonyl)amino]phenyl}-2-hydroxyethyl)amino]-2-methylpropyl}phenyl)-N-[(4′-hydroxybiphenyl-3-yl)methyl]-acetamide, dibenzoyl-(L)-tartrate salt

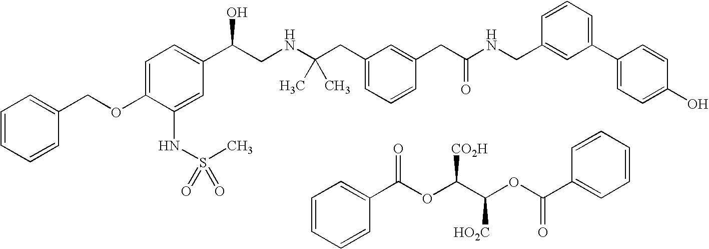

A solution of dibenzoyl-(L)-tartaric acid (8.74 g; 24.4 mmol) in 2-butanone (19 mL) was added to the n-butyl acetate solution of the amine from preparation 22 (alternative process 3), giving a thick gum. The mixture was diluted with further 2-butanone (76 mL) and warmed to 40° C., giving an orange solution. This orange solution was then cooled to ambient temperature and added to tert-butyl methyl ether (285 mL) over 15 min with vigorous stirring at ambient temperature, rinsing with 2-butanone (2×9.5 mL) and the resulting slurry was granulated for 18 h at ambient temperature. The solid was isolated by filtration, washing with further tert-butyl methyl ether (2×95 mL) to give the title compound (24.64 g) as an off-white solid (estimated as an approximately 4:3 mixture of the amine and acid components, and containing some tert-butyl methyl ether).

¹H NMR (DMSO-d₆, 400 MHz) δ: 1.02 (s, 6H), 2.70-3.10 (m, 6H), 3.40 (s, 3H), 4.25 (d, 2H), 4.65 (br. d, 1H), 5.18 (s, 2H), 5.60 (s, 1.5H), 6.81 (d, 2H), 6.90-7.60 (m, 23.5H), 7.90 (m, 2H), 8.55 (t, 1H).

### Alternative Process for the Preparation of the Product of Preparation 22a

A solution of dibenzoyl-(L)-tartaric acid (9.0 g; 25.1 mmol) in 2-butanone (50 mL) was added to a solution of the product of preparation 22 (16.9 g) in butyronitrile (35 mL). A further 50 mL 2-butanone was added to fully dissolve all the material. This solution was then added to tert-butyl methyl ether (500 mL) over 10 min with vigorous stirring at ambient temperature, rinsing with further 2-butanone (20 mL). Additional tert-butyl methyl ether (100 mL) was added to the slurry and this was then aged at ambient temperature for 3 h. The solid was isolated by filtration and washed with tert-butyl methyl ether (200 mL) to give the title compound as a yellow solid (20.86 g).

### Preparation 23

### 2-(3-{2-[((2R)-2-{4-Benzyloxy-3-[(dimethylsulfonyl)amino]-2-phenyl}-2-{[tert-butyl(dimethyl)silyl]oxy}ethyl)amino]-2-methylpropyl}phenyl)-N-[(4′-hydroxybiphenyl-3-yl)methyl]-acetamide

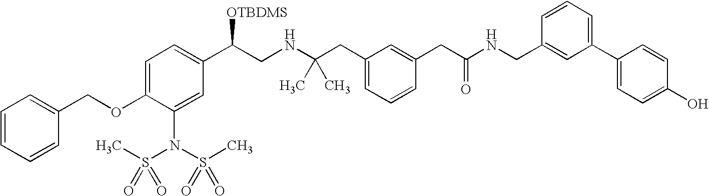

A mixture of the protected bromohydrin from preparation 8 (1.0 g; 1.7 mmol), the amine from preparation 20 (650 mg; 1.67 mmol) and sodium hydrogen carbonate (560 mg; 6.7 mmol) in butyronitrile (2 mL) was heated at reflux for 31 h under an inert atmosphere. The reaction mixture was then cooled to ambient temperature and diluted with propionitrile (10 mL) and water (10 mL). The phases were separated; the organic phase was dried with anhydrous magnesium sulfate, filtered and concentrated to give the title compound (1.54 g), which was used directly in the next step without purification.

### Preparation 24

### 2-(3-{2-[((2R)-2-{4-Benzyloxy-3-[(methylsulfonyl)amino]phenyl}-2-{[tert-butyl(dimethyl)silyl]oxy}ethyl)amino]-2-methylpropyl}phenyl)-N-[(4′-hydroxybiphenyl-3-yl)methyl]-acetamide

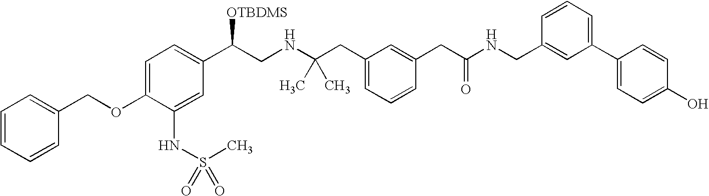

The crude bis-sulfonamide from preparation 23 (1.54 g; assume 1.7 mmol) was dissolved in ethanol (5 mL). Water (5 mL) and sodium hydroxide (600 mg; 15 mmol) were added and the resulting mixture was stirred at ambient temperature for 72 h. The mixture was then acidified to pH 1 with concentrated hydrochloric acid, and then neutralised (to pH 10) with aqueous ammonia (35%). The product was then extracted into propionitrile (2×20 mL). The combined organic extracts were dried with anhydrous magnesium sulfate, filtered and the solution concentrated in vacuo to yield the title compound as a yellow oil (1.24 g) which was used without purification in the next step.

### Preparation 25

### 2-(3-{2-[((2R)-2-{4-Benzyloxy-3-[(methylsulfonyl)amino]phenyl}-2-{[tert-butyl(dimethyl)silyl]oxy}ethyl)amino]-2-methylpropyl}-3-phenyl)-N-[(4′-hydroxybiphenyl-3-yl)methyl]-acetamide

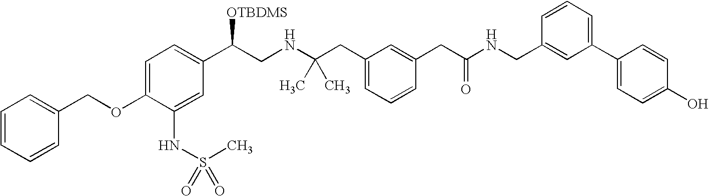

A mixture of the protected bromohydrin from preparation 7 (2.0 g; 3.92 mmol), the amine from preparation 20 (1.5 g; 3.86 mmol) and sodium hydrogen carbonate (1.0 g; 11.9 mmol) in butyronitrile (4 mL) was refluxed for about 30 h under an inert atmosphere. The cooled reaction mixture was then diluted with propionitrile (20 mL), washed with water (2×10 mL), dried with anhydrous magnesium sulfate, filtered and concentrated in vacuo to yield the title product (3.05 g, 80% pure), which was used in the next step without further purification.

### Preparation 26

### N-[(4′-Hydroxybiphenyl-3-yl)methyl]-2-(3-{2-[((2R)-2-hydroxy-2-{4-hydroxy-3-[(methylsulfonyl)amino]phenyl}ethyl)amino]-2-methylpropyl}phenyl)acetamide

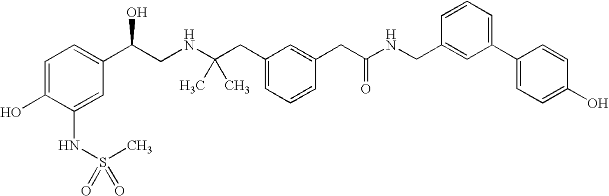

Palladium hydroxide (20 wt % on carbon; 60 mg) was added to a solution of the benzyl ether from preparation 22 (613 mg; 0.87 mmol) in a mixture of ethanol (4.5 mL) and water (1.5 mL). This mixture was placed under a hydrogen atmosphere (60 psi) and was stirred at 60° C. for 18 h. The reaction mixture was then purged with nitrogen and diluted with aqueous ammonia (35%) in ethanol (1:9, ˜15 mL), filtered through Celite, washing with further aqueous ammonia (35%) in ethanol (1:9, ˜15 mL) and ethanol (˜10 mL). The liquors were concentrated to a residue and then dissolved in a mixture of aqueous ammonia (35%) and THF (1:19, ˜10 mL) and filtered through a silica pad, washing with further aqueous ammonia (35%)/THF (1:19, ˜250 mL). The liquors were concentrated to a residue, slurried in refluxing methanol (10 mL), then cooled to ambient temperature and stirred for 18 h. The precipitate was isolated by filtration, washing with methanol to give the title compound (296 mg) as an off white solid.

¹H NMR (DMSO-d₆, 400 MHz) δ: 0.88 (s, 3H), 0.90 (s, 3H), 2.54 (s, 2H), 2.62 (m, 2H), 2.88 (s, 3H), 3.44 (s, 2H), 4.30 (d, 2H), 4.41 (dd, 1H), 6.81 (m, 3H), 6.98 (m, 2H), 7.05-7.18 (m, 5H), 7.25-7.42 (m, 5H), 8.49 (t, 1H) ppm.

### Alternative Processes for the Preparation of the Product of Preparation 26

Alternative Process 1:

A mixture of the salt from preparation 22a (6.7 g; 6.74 mmol), tetrahydrofuran (67 mL) and concentrated aqueous ammonia (10 mL) was stirred vigorously for 15 min. The phases were separated and the organic phase was washed with a mixture of water (10 mL) and saturated brine (10 mL). The tetrahydrofuran solution was then distilled at constant volume (50-60 mL), adding additional tetrahydrofuran as required, until a total of 60 mL of distillate had been collected. The solution was then diluted with further tetrahydrofuran (total volume approx 84 mL) and water (18 mL) and palladium on carbon (5%, 50% water wet; 670 mg) was added and the resulting mixture was hydrogenated at 40° C./50 psi hydrogen pressure for 31 h, with further catalyst (500 mg and 600 mg) being added after 8 h and 24 h respectively. The mixture was removed from the hydrogenation reactor and Arbocel (5 g) was added and the mixture was stirred for 20 min. The resulting slurry was filtered through a pad of Arbocel, washing with tetrahydrofuran/water (9:1, approx 50 mL). The filtrate was then diluted with acetonitrile (85 mL) and the tetrahydrofuran was removed by distillation. Once the vapour temperature reached 76° C., a further 20 mL acetonitrile was added and then a further 20 mL of distillate was collected. The resulting slurry was cooled to ambient temperature and aged for 16 h. The solid was collected by filtration, washing with acetonitrile-water (9:1, 40 mL) and dried under vacuum for 20 min. The damp-cake was then slurried in methanol-water (9:1, 40 mL), initially at 50° C. for 1 h, and then at ambient temperature for 16 h. The precipitate was isolated by filtration, washing with methanol-water (8:2, 40 mL) to give the title compound as an off-white solid (2.25 g; 54%).

Alternative Process 2:

A mixture of the salt from preparation 22a (11.26 g; 11.6 mmol), 2-methyltetrahydrofuran (100 mL), concentrated aqueous ammonia (50 mL) and water (150 mL) was stirred vigorously for 1 h. The phases were separated and the aqueous was back-extracted with 2-methyltetrahydrofuran (20 mL). The combined organic phase was washed with water (50 mL) and then diluted with ethylene glycol (100 mL) and the 2-methyltetrahydrofuran was removed by distillation under reduced pressure. Palladium on carbon catalyst (5%, 50% water wet; 1100 mg) was added and the resulting mixture was hydrogenated at 40° C./50 psi hydrogen pressure for 18 h. The mixture was removed from the hydrogenation reactor and Arbocel (5 g) was added. The resultant mixture was stirred for 30 min and then filtered through a pad of Arbocel, washing with ethylene glycol (25 mL). Fresh palladium on carbon catalyst (5%, 50% water wet; 1100 mg) was added and the resulting mixture was hydrogenated at 40° C./50 psi hydrogen pressure for 7 h and then at 40° C./80 psi for 16 h. Additional palladium on carbon (5%, 50% water wet; 1000 mg) was then added and the resulting mixture was hydrogenated at 40° C./80 psi hydrogen pressure for 4 h. The mixture was removed from the hydrogenation reactor and Arbocel (10 g) was added. The resultant mixture was stirred for 30 min and then filtered through a pad of Arbocel, washing with ethylene glycol (25 mL). The ethylene glycol filtrate was then added to water (200 mL) over approximately 10 min with vigorous stirring, washing with additional ethylene glycol (20 mL) and water (100 mL) and the resulting light brown slurry was stirred at ambient temperature for 30 min. The solid was isolated by filtration, washing with water (100 mL) and dried at 40° C. in a vacuum oven. The resulting light-brown solid was further purified by slurrying in methanol-water (9:1, 54 mL), initially at 50° C. for 2 h, and then at ambient temperature for 16 h. The precipitate was isolated by filtration, washing with methanol-water (8:2, 15 mL) to give the title compound as an off-white solid (4.57 g; 64%).

Alternative Process 3:

A mixture of the protected bromohydrin from preparation 7 (10.93 g; 21.2 mmol), the amine from preparation 20 (7.50 g; 19.3 mmol) and sodium hydrogen carbonate (9.0 g; 107.1 mmol) in n-butyl acetate (55 mL) was refluxed under nitrogen for 53 h. The mixture was cooled to ambient temperature and diluted with water (180 mL) and ethyl acetate (180 mL). The phases were separated and the organic phase was washed successively with 1M aqueous (L)-tartaric acid (55 mL), water (55 mL), water-concentrated aqueous ammonia (3:1, 60 mL) and water (55 mL). Palladium on carbon catalyst (5%, 50% water wet; 1300 mg) was added and the resulting mixture was hydrogenated at 60° C./60 psi hydrogen pressure for 24 h. The reaction mixture was removed from the hydrogenation reactor and Arbocel (13 g) was added and the resulting slurry was stirred for 30 min. The mixture was then filtered through a pad of Arbocel and the catalyst bed was washed with ethyl acetate (200 mL). The pale yellow filtrate was then concentrated under reduced pressure to remove the ethyl acetate, methanol (60 mL) was then added and the mixture was concentrated to dryness under reduced pressure. The resulting viscous orange-brown oil was dissolved in methanol (100 mL) and placed in a polypropylene vessel. Ammonium fluoride (2.1 g; 56.7 mmol) was added, washing with water (20 mL) and methanol (20 mL) and the resulting solution was stirred at ambient temperature for 65 h. The precipitated solid was isolated by filtration, washing with methanol-water (8:2, 100 mL) and dried under suction for 10 min and at 40° C. in a vacuum oven for 4 h. The pale brown solid was then slurried in methanol-water (9:1, 75 mL), initially at 50° C. for 2 h, and then at ambient temperature for 16 h. The precipitate was isolated by filtration, washing with methanol-water (8:2, 2×20 mL) and dried at 40° C. in a vacuum oven, the solid was then further purified by slurrying in water (80 mL) at ambient temperature for 16 h. The solid was then isolated by filtration and washed with water (50 mL) to give the title compound (6.01 g; 50%) as an off-white solid.

1. A process for preparing a compound of formula (I) 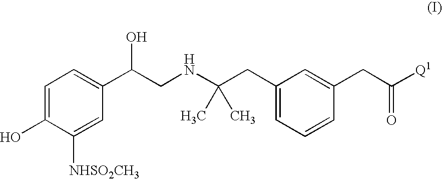
wherein Q¹ is a group selected from: 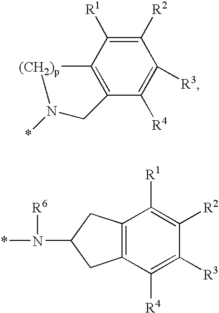
and a group *-NR⁶-Q²-A, wherein the symbol * represent the attachment point to the carbonyl group, p is 1 or 2, Q² is a C₁-C₄ alkylene optionally substituted with one hydroxy group, R⁶ is H or C₁-C₄ alkyl and A is pyridyl optionally substituted with OH, C₃-C₇ cycloalkyl optionally substituted with OH, or a group 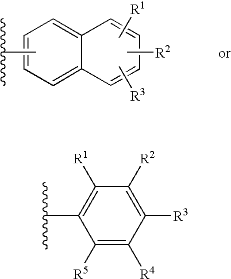
wherein R¹, R², R³, R⁴ and R⁵ are the same or different and are selected from H, C₁-C₄ alkyl, OR⁷, SR⁷, halo, CN, CF₃, OCF₃, COOR⁷, SO₂NR⁷R⁸, CONR⁷R⁸, NR⁷R⁸, NHCOR⁷ and phenyl optionally substituted with 1 to 3 groups selected from OR⁷, halo and C₁-C₄ alkyl, wherein R⁷ and R⁸ are the same or different and are selected from H or C₁-C₄ alkyl;
comprising
(a) reacting a compound of formula 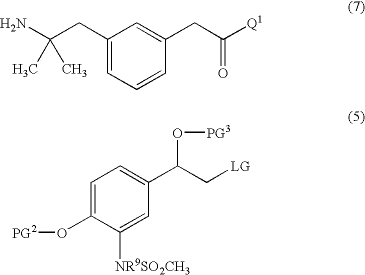
with a compound of formula (5),
wherein PG² is a suitable phenol protecting group, PG³ is a suitable hydroxyl protecting group, LG is a suitable leaving group and R⁹ is H or SO₂CH₃ to form a compound of formula 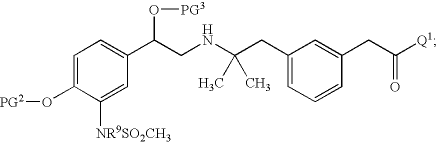
(b) reacting said compound of formula 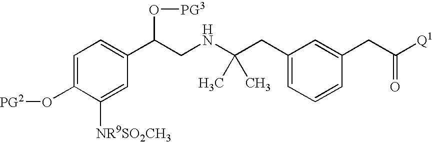
with a hydroxy deprotecting agent to form a compound of formula 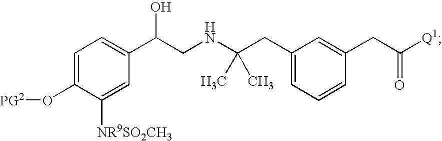
(c) when R⁹ is SO₂CH₃, reacting said compound of formula 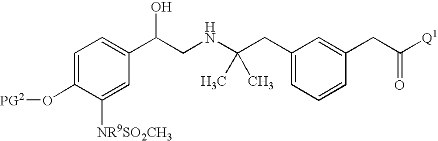
with a n amine deprotecting agent to form a compound of formula (2) 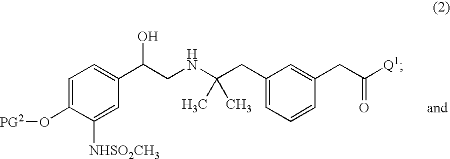
(d) reacting said compound of formula (2) 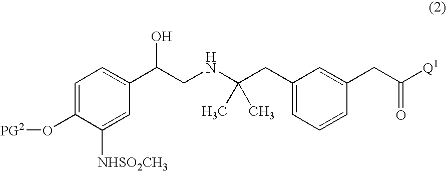
with a phenol deprotecting agent to form said compound of formula (I).

2-5. (canceled)

6. A process for preparing a compound of formula (3a) 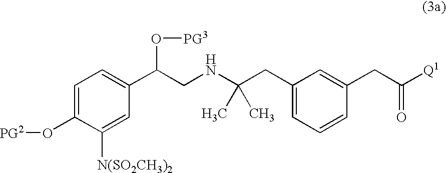
wherein PG² is a suitable phenol protecting group, PG³ is a suitable hydroxyl protecting group, Q¹ is a croup selected from: 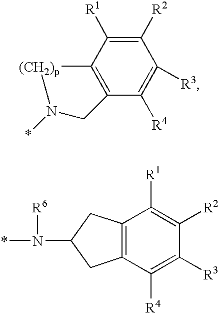
and a group *-NR⁶-Q²-A, wherein the symbol * represent the attachment point to the carbonyl group, p is 1 or 2, Q² is a C₁-C₄ alkylene optionally substituted with one hydroxy group, R⁶ is H or C₁-C₄ alkyl and A is pyridyl optionally substituted with OH, C₃-C₇ cycloalkyl optionally substituted with OH, or a group 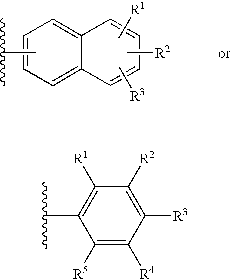
wherein R¹, R², R³, R⁴ and R⁵ are the same or different and are selected from H, C₁-C₄ alkyl. OR⁷, SR⁷, halo, CN, CF₃, OCF₃, COOR⁷, SO₂NR⁷R⁸, CONR⁷R⁸, NR⁷R⁸, NHCOR⁷ and phenyl optionally substituted with 1 to 3 groups selected from OR⁷, halo and C₁-C₄ alkyl, wherein R⁷ and R⁸ are the same or different and are selected from H or C₁-C₄ alkyl;
comprising reacting a compound of formula (7) 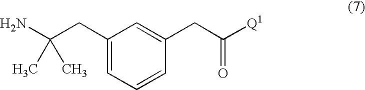
with a compound of formula (5) 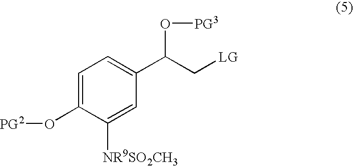
wherein LG is a suitable leaving group and R⁹ is SO₂CH₃ to obtain said a compound of formula (3a)

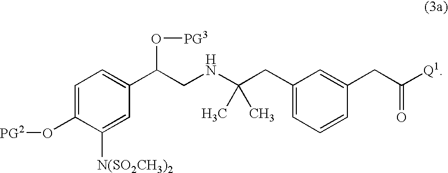

7-9. (canceled)

10. A process for preparing a compound of formula (4) 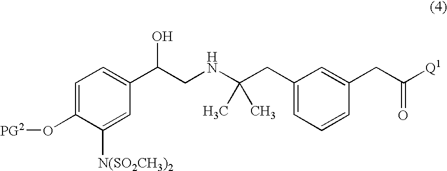
wherein PG² is a suitable phenol protecting group;
Q¹ is a group selected from: 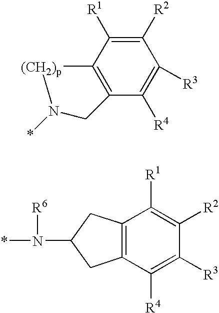
and a group *-NR⁶-Q²-A, wherein the symbol * represent the attachment point to the carbonyl group, p is 1 or 2, Q² is a C₁-C₄ alkylene optionally substituted with one hydroxy group, R⁶ is H or C₁-C₄ alkyl and A is pyridyl optionally substituted with OH, C₃-C₇ cycloalkyl optionally substituted with OH, or a group 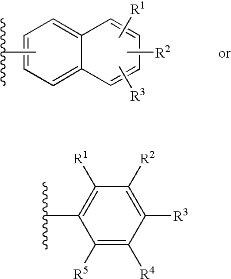
wherein R¹, R², R³, R⁴ and R⁵ are the same or different and are selected from H, C₁-C₄ alkyl, OR⁷, SR⁷, halo, CN, CF₃, OCF₃, COOR⁷, SO₂NR⁷R⁸, CONR⁷R⁸, NR⁷R⁸, NHCOR⁷ and phenyl optionally substituted with 1 to 3 groups selected from OR⁷, halo and C₁-C₄ alkyl, wherein R⁷ and R⁸ are the same or different and are selected from H or C₁-C₄ alkyl;
comprising of reacting a compound of formula (7) 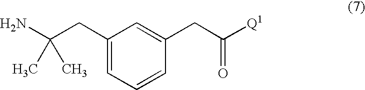
with a compound of formula (6) 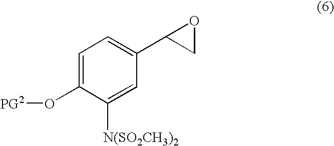
to obtain said a compound of formula (4)

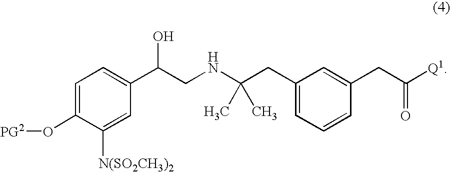

11-13. (canceled)

14. A process of claim 1 where LG is bromide.

15. A process of claim 1 where PG³ is TBDMS.

16. A process of claim 1 where PG² is benzyl.

17-27. (canceled)

28. A process for preparing a compound of formula (16) 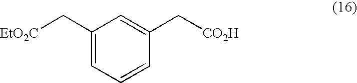
comprising hydrolyzing a compound of formula (18) 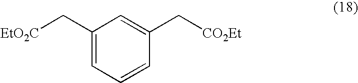
in the presence of an enzyme selected from a lipase, an esterase or a protease.

29. A process according to claim 28 where said enzyme is selected from Mucor Miehei esterase, Rhizomucor Miehei lipase, Thermomuces Languinosus lipase, and Penicillin acylase.

30. A process according to claim 29 where said enzyme is Thermomuces Languinosus lipase.

31. A process according to claim 28 where the reaction is carried out at a pH between 5 and 9 and at a temperature between 10° C. and 40° C. in water, in the presence of a suitable buffering agent, and optionally in the presence of a suitable base.

32. A process according to claim 1 where Q¹ is a group of formula

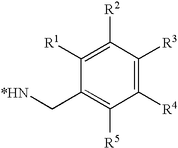

33. A process according to claim 32 where R¹, R², R³, R⁴ and R⁵ are the same or different and are selected from H, C₁-C₄ alkyl, OR⁶, SR⁶, halo, CF₃, OCF₃, SO₂NR⁶R⁷, CONR⁶R⁷, NR⁶R⁷, NHCOR⁷, provided at least two of R¹ to R⁵ are H;
and R⁶ and R⁷ are the same or different and are selected from H or C₁-C₄ alkyl.

34. A process according to claim 32 where R¹, R², R³, R⁴ and R⁵ are the same or different and are selected from H, OH, CH₃, OCH₂—CH₃, SCH₃, halo, CF₃, OCF₃, provided at least two of R¹ to R⁵ are H.

35. A process according to claim 32 where one of R¹ to R⁵ is OH.

36. A process according to claim 32 where one of R¹, R², R³, R⁴ and R⁵ is phenyl substituted by OH and the others are H.

37. A process according to claim 32 where R² is 4-hydroxy-phenyl and R¹, R³, R⁴ and R⁵ are H.

38. A process according to claim 32 where R² and R³ are Cl and R¹, R⁴ and R⁵ are H.

39. A process according to claim 33 where the asymmetric carbon substituted with an hydroxyl or a OPG³ group is in R configuration.

40. A compound of formula 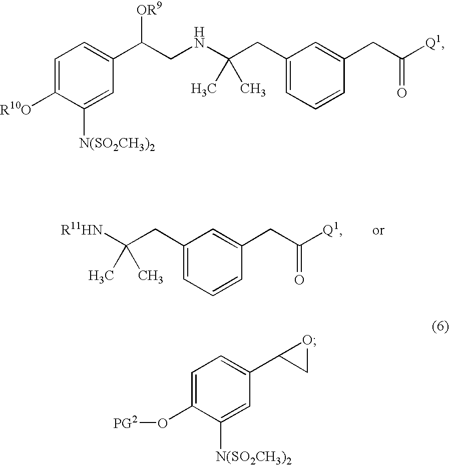
wherein Q¹ is a group selected from: 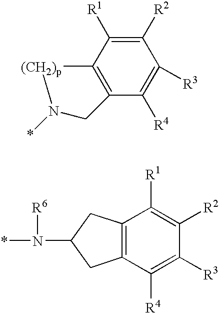
and a group *-NR⁶-Q²-A, wherein the symbol * represent the attachment point to the carbonyl group, p is 1 or 2, Q² is a C₁-C₄ alkylene optionally substituted with one hydroxy group, R⁶ is H or C₁-C₄ alkyl and A is pyridyl optionally substituted with OH, C₃-C₇ cycloalkyl optionally substituted with OH, or a group 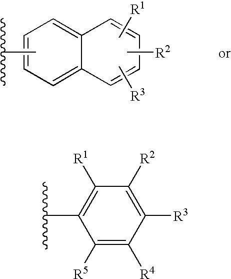
wherein R¹, R², R³, R⁴ and R⁵ are the same or different and are selected from H, C₁-C₄ alkyl, OR⁷, SR⁷, halo, CN, CF₃, OCF₃, COOR⁷, SO₂NR⁷R⁸, CONR⁷R⁸, NR⁷R⁸, NHCOR⁷ and phenyl optionally substituted with 1 to 3 groups selected from OR⁷, halo and C₁-C₄ alkyl, wherein R⁷ and R⁸ are the same or different and are selected from H or C₁-C₄ alkyl;
R¹⁰ is H or PG² where PG² is a suitable phenol protecting group, R⁹ is H or PG³ where PG³ is a suitable hydroxyl protecting group, and R¹¹ is H or PG¹ where PG¹ is a suitable amino protecting group.

41. (canceled)

42. A compound of claim 40 of formula 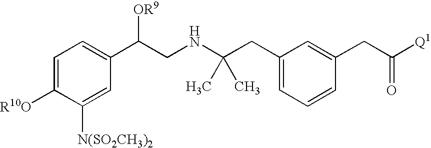
where the asymmetric carbon substituted with OR⁹ is in the R configuration.

43. A compound of claim 40 of formula 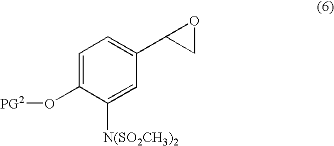
wherein PG² is benzyl.

44. A compound according to claim 43, said compound being of formula:

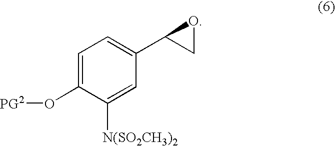

45. A compound of claim 40 of formula 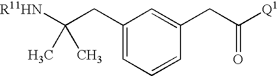
wherein R¹¹ is H.

46. A compound according to claim 40, said compound being selected from
2-(3-{2-[((2R)-2-{4-Benzyloxy-3-[(dimethylsulfonyl)amino]phenyl}-2-{[tert-butyl(dimethyl)silyl]oxy}ethyl)amino]-2-methylpropyl}phenyl)-N-[(4′-hydroxybiphenyl-3-yl)methyl]-acetamide;
2-(3-{2-[((2R)-2-{4-Benzyloxy-3-[(dimethylsulfonyl)amino]phenyl}-2-hydroxyethyl)amino]-2-methylpropyl}phenyl)-N-[(4′-hydroxybiphenyl-3-yl)methyl]-acetamide;
tert-butyl-[2-(3-{[(4′-hydroxybiphenyl-3-ylmethyl)-carbamoyl]-methyl}-phenyl)-1,1-(dimethyl)ethyl]carbamate;
2,2,2-Trichloro-N-[2-(3-{[4′-hydroxy-biphenyl-4-ylmethyl)-carbamoyl]-methyl}-phenyl)-1,1-dimethyl-ethyl]acetamide,
2-Chloro-N-{2-[3-(2-{[(4′-hydroxybiphenyl-3-yl)methyl]amino}-1-2-oxoethyl)phenyl]-1,1-dimethylethyl}acetamide,
2-[3-(2-Amino-2-methylpropyl)-phenyl]-N-[(4′-hydroxybiphenyl-3-yl)methyl]acetamide, and N—[(R)-2-benzyloxy-5-oxiranyl-phenyl]-dimethanesulfonamide.
